(19) United States
(12) Patent Application Publication
(10) Pub. No.: US 2023/0116038 A1
(43) Pub. Date: Apr. 13, 2023

(54) STEERABLE CATHETERS AND RELATED METHODS

(71) Applicant: Deinde Medical Corp., Miramar, FL (US)

(72) Inventors: Robert C. Farnan, Fort Lauderdale, FL (US); Scott Arp, Miami, FL (US); Peter Kratsch, Leander, TX (US)

(21) Appl. No.: 18/080,995

(22) Filed: Dec. 14, 2022

Related U.S. Application Data
(63) Continuation of application No. PCT/US2021/037803, filed on Jun. 17, 2021.
(60) Provisional application No. 63/041,395, filed on Jun. 19, 2020.

(51) Int. Cl. A61M 25/01; A61B 18/14; A61M 29/02; A61N 5/06; A61F 2/95

(52) U.S. Cl. CPC A61M 25/0136; A61B 18/1492; A61M 29/02; A61N 5/0603; A61F 2/9517

(57) ABSTRACT

A steerable catheter, such as a dilator, includes an elongated shaft including a proximal end portion and a distal end portion, an end effector disposed at the distal end portion of the shaft, and a handle disposed at the proximal end portion of the shaft. The end effector includes a dilation portion disposed proximate the distal end portion of the shaft, a transition portion disposed distally on the dilation portion, and a distal tip portion disposed distally on the transition portion. The tip portion is movable relative to the dilation portion. The handle includes a tip portion steering actuator enabling a user to steer the tip portion. The tip portion has a tip portion diameter. The dilation portion has a dilation portion diameter which is greater than the tip portion diameter. The transition portion tapers from the tip portion diameter to the dilation portion diameter.

### CROSS REFERENCE TO RELATED APPLICATION

This application is a continuation of PCT Application Ser. No. PCT/US2021/037803 filed Jun. 17, 2021, which claims the priority of U.S. Provisional Patent Application Ser. No. 63/041,395 filed Jun. 19, 2020, the disclosures of which are incorporated herein by reference in their entirety.

### TECHNICAL FIELD

The present disclosure generally relates to minimally invasive surgical devices and, more particularly, to steerable catheters. Specifically, the disclosure relates to steerable catheters for creating openings in tissues and/or for Photobiomodulation (PBMT), during minimally invasive neurosurgical procedures, and related methods.

### BACKGROUND

The present disclosure contemplates that treatments for some conditions may include surgically creating openings through tissues (e.g., biological tissue membranes). For example, for patients suffering from non-communicating hydrocephalus, the normal channel for drainage of the cerebral spinal fluid (“CSF”) from the third ventricle to the lower CSF chambers and central canal that leads to the spinal cord is blocked by an occlusion within the cerebral aqueduct. These patients, through the normal production of the CSF, experience a “rise” in the pressure of the lamina and third ventricles, causing these fluid chambers to distend. To relieve the pressure, neurosurgeons have used a procedure called a third ventriculostomy to rupture the lamina terminalis. This procedure requires creating a hole through the patient's skull and moving brain tissue to allow the introduction and navigation of an endoscope to the desired site for creation of the ventriculostomy. Once the lamina terminalis is visible in the endoscopic image, a balloon catheter (e.g., Fogarty) is advanced up to and through the membrane. Once the balloon portion of the catheter is passed through the membrane, it is inflated and then retracted through the membrane to create the fenestration (e.g., hole) between the CSF chambers. An alternative ventriculostomy procedure uses similar access through the skull and brain, but, instead of the endoscope/balloon catheter combination, a surgical instrument is used that discretely cauterizes the membrane as it is opened is utilized. While such procedures have been successfully used on patients, these procedures involve risks and disadvantages associated with surgically penetrating the skull, dura mater, and/or brain tissues.

The present disclosure further contemplates that treatments for some conditions may include Photobiomodulation (PBMT) which is the application of red and near infra-red light for medical treatment. The light can originate from a laser or a light emitting diode (LED), and can be pulsed and/or continuous. The health benefits of PBMT are well documented with the application of light typically to the exterior of the body of the patient in a non-invasive manor. Generally, transcranial PBMT procedures involve the placement of a light source, or multiple light sources, on one or more areas of the patient's head, with the goal of treating a certain part of the brain. In transcranial PBMT, light must pass through layers of tissue including the scalp, periosteum, skull bone, meninges, and dura, before reaching the cortical surface of the brain. Due to the exponential attenuation of light during the journey through the skull and brain tissues, only a small fraction of the incident light may be delivered to the intended tissue. A device and method of delivering PBMT more directly to the tissue to be treated, and specifically the brain and/or spinal tissue, may provide unique benefits.

Accordingly, and despite the various advances already made in this field, there is a need for further improvements related to steerable catheters for many desired uses during surgical procedures.

### SUMMARY

Generally, a steerable dilator is provided and includes an elongated shaft with a proximal end portion and a distal end portion, an end effector disposed at the distal end portion of the shaft, and a handle disposed at the proximal end portion of the shaft. The end effector includes a proximal dilation portion disposed proximate the distal end portion of the shaft, a transition portion disposed distally on the dilation portion, and a distal tip portion disposed distally on the transition portion. The handle includes a tip portion steering actuator disposed on the handle. The tip portion has a tip portion diameter. The dilation portion has a dilation portion diameter. The dilation portion diameter is greater than the tip portion diameter. The transition portion tapers from the tip portion diameter to the dilation portion diameter between the tip portion and the dilation portion. The tip portion is movable relative to the dilation portion, and the tip portion steering actuator enables a user to steer the tip portion by moving the tip portion with the tip portion steering actuator.

In some embodiments, the diameter of the tip portion may be equal to the diameter of the dilation portion. The transition portion may taper generally smoothly from the tip portion diameter to the dilation portion diameter. The transition portion may taper generally linearly from the tip portion diameter to the dilation portion diameter. The diameter of the shaft may be approximately equal the dilation portion diameter. As used herein, the term steerable dilator, or simply dilator, includes steerable catheters with a dilation mechanism. It should be understood that some embodiments described herein may be used with steerable catheters without a dilation mechanism.

In some embodiments, the tip portion may be steerable relative to the dilation portion in a tip portion steering plane. A first tip portion steering line may operatively connect the tip portion and the tip portion steering actuator enabling a user to steer the tip portion in a first direction in the tip portion steering plane by moving the tip portion steering actuator in a first tip portion steering actuator direction. The tip portion may include a steering ring disposed on the tip portion. The first tip portion steering line may operatively connect the tip portion steering ring and the tip portion steering actuator enabling a user to steer the tip portion in the first direction in the tip portion steering plane. The tip portion steering actuator may include a toothed portion engaged with and operative to rotate a toothed gear, and the first tip portion steering line may be coupled to the toothed gear. The dilator may include a second tip portion steering line operatively connecting the tip portion and the tip portion steering actuator enabling a user to steer the tip portion in a second direction, the second direction being generally opposite the first direction in the tip portion steering plane, by moving the tip portion steering actuator in a second tip portion steering actuator direction. The second tip portion steering line may operatively connect the tip portion steering ring and the tip portion steering actuator enabling a user to steer the tip portion in the second direction. The first tip portion steering line may be coupled to the toothed gear of the tip portion steering actuator. The first and/or second tip portion steering line may include at least one of metal wire and suture material.

In alternative or additional aspects, the dilation portion may be steerable relative to the shaft in a dilation portion steering plane. The handle may include a dilation portion steering actuator disposed on the handle. The dilation portion may include a first dilation portion steering line operatively connecting the dilation portion and the dilation portion steering actuator enabling a user to steer the dilation portion in a first direction in the dilation portion steering plane, by moving the dilation portion steering actuator in a first dilation portion steering actuator direction. The dilation portion may include a dilation steering ring disposed on the dilation portion. The first dilation portion steering line may operatively connect the dilation portion steering ring and the dilation portion steering actuator enabling a user to steer the dilation portion in the first direction in the dilation portion steering plane. The dilation portion steering actuator may include a toothed portion engaged with and operative to rotate a toothed gear, and the first dilation portion steering line may be coupled to the toothed gear. The dilator may include a second dilation portion steering line operatively connecting the dilation portion and the dilation portion steering actuator enabling a user to steer the dilation portion in a second direction, the second direction being generally opposite the first direction in the dilation portion steering plane, by moving the dilation portion steering actuator in a second dilation portion steering actuator direction. The second dilation portion steering line may operatively connect the dilation portion steering ring and the dilation portion steering actuator enabling a user to steer the dilation portion in the second direction. The first and/or second dilation portion steering line may include at least one of metal wire and suture material.

In alternative embodiments, the tip portion may be steerable relative to the dilation portion in a tip portion steering plane. The dilation portion may be steerable relative to the shaft in a dilation portion steering plane. The tip portion steering plane and the dilation portion steering plane may be substantially parallel. The dilation portion steering plane may be disposed at a steering plane angle relative to the tip portion steering plane, the steering plane angle being greater than zero. In some embodiments the steering plane angle may be about 90 degrees. The dilation portion steering plane may be substantially perpendicular to the tip portion steering plane.

In alternative or additional aspects, the dilator may include a central lumen extending longitudinally through the handle, the shaft, the dilation portion, the transition portion, and the tip portion. The central lumen may be configured to receive a guidewire therethrough. The distal end of the tip portion may be generally rounded. In alternate embodiments, the distal end of the tip portion may be generally pointed.

In some embodiments, the shaft may be generally flexible. The stiffness of the shaft may vary over a length of the shaft between the proximal end portion and the distal end portion. The stiffness of the shaft may vary in discrete intervals over the length of the shaft. The stiffness of the shaft may be greater proximate the proximal end portion than proximate the distal end portion. The shaft of the dilator may include an inner layer, an intermediate layer, and an outer layer.

The steerable dilator may include a tip portion energizing element disposed on the tip portion. The tip portion energizing element may be configured to be operatively connected to a source of electrical energy. The source of electrical energy may be configured to deliver at least one of radio frequency energy and electrocautery energy to the tip portion energizing element. The dilator may include a wiring harness extending from the tip portion energizing element, through a lumen in the shaft, and through the handle, for example. The dilator may include a dilation portion energizing element disposed on the dilation portion. The dilation portion energizing element may be configured to be operatively connected to a source of electrical energy. The source of electrical energy may be configured to deliver at least one of radio frequency energy and electrocautery energy to the dilation portion energizing element. The dilator may include a wiring harness extending from the dilation portion energizing element, through a lumen in the shaft, and through the handle, for example. The steerable dilator may be part of a dilator system, including the dilator and an electrosurgical generator, the electrosurgical generator comprising the source of electrical energy.

In some embodiments, the dilator may include an expandable element disposed on the dilation portion. The expandable element may include an inflatable element. The inflatable element may be generally toroidal when inflated. The inflatable element may be generally cylindrical when deflated, for example.

In an alternative embodiment, a steerable dilator is provided and includes an elongated shaft with a proximal end portion and a distal end portion, an end effector disposed at the distal end portion of the shaft, and a handle disposed at the proximal end portion of the shaft. The end effector includes a dilation portion disposed proximate the distal end portion of the shaft, and a distal tip portion disposed distally on the dilation portion. The handle includes a steering actuator rotatably disposed on the handle. The tip portion is movable relative to the dilation portion. The dilation portion is movable relative to the shaft. The steering actuator enables a user to steer the tip portion and the dilation portion by moving the tip portion and the dilation portion with the steering actuator.

In alternative embodiments, the steerable dilator may include a steering actuator rotatably disposed on the handle. The steering actuator includes, a yoke rotatably disposed on the handle, an actuator body rotatably disposed on the yoke, a rotational actuator rotatably disposed on the actuator body, and an actuator handle disposed on the actuator body. The actuator handle enables a user to operate the steering actuator. The steering actuator may include one or more steering line attachment points. The steering actuator may be generally configured as one or more pivoted supports that allow for the rotation of the steering actuator about a first axis of rotation and a second axis of rotation relative to the handle. Moving the actuator handle in a first actuator direction may rotate the steering actuator about a first actuator axis of rotation. Moving the actuator handle in a second actuator direction may rotate the steering actuator body about the first actuator axis of rotation, the second actuator direction being generally opposite the first actuator direction. The yoke may include a series of teeth arranged to engage an opposed series of teeth on the rotational actuator. Moving the actuator handle in a third direction may rotate the steering actuator body about a second actuator axis of rotation. Rotating the steering actuator body about a second actuator axis of rotation may cause the teeth on the yoke to apply a force to the teeth on the rotational actuator which may cause the rotational actuator to rotate about a third actuator axis of rotation. Moving the actuator handle in a fourth direction may rotate the steering actuator body about a second actuator axis of rotation, the fourth direction being generally opposite the third direction. Rotating the steering actuator body about a second actuator axis of rotation may cause the teeth on the yoke to apply a force to the teeth on the rotational actuator which may cause the rotational actuator to rotate about a third actuator axis of rotation. The rotational actuator may be configured for one handed operation where the user may grip the handle with one hand and move the actuator handle with the thumb of said hand, for example.

In some embodiments, the first tip portion steering line may be coupled to the steering actuator and moving the actuator handle in a first actuator direction may steer the tip portion in the first direction in the first tip portion steering plane. The second tip portion steering line may be coupled to the steering actuator and moving the actuator handle in a second actuator direction may steer the tip portion in the second direction in the first tip portion steering plane. The first dilation portion steering line may be coupled to the steering actuator and moving the actuator handle in a third actuator direction may steer the dilation portion in the first direction in the first dilation portion steering plane. The second dilation portion steering line may be coupled to the steering actuator and moving the actuator handle in a fourth actuator direction may steer the dilation portion in the second direction in the first dilation portion steering plane.

In alternative or additional aspects, the dilator may include an expandable element disposed on the dilation portion. The expandable element on the dilation portion may include an inflatable portion. The expandable element on the dilation portion may include a radiopaque material. The inflatable portion on the dilation portion may be generally cylindrical when not inflated and generally toroidal when inflated, for example. The inflatable portion on the dilation portion may include other shapes when inflated.

In some embodiments, the tip portion may include an expandable element disposed on the tip portion. The expandable element on the tip portion may include an inflatable element. The expandable element on the tip portion may include a radiopaque material. The inflatable element on the tip portion may generally mimic the shape of the tip when not inflated and may be generally teardrop shaped when inflated, for example. The inflatable portion on the tip portion may include other shapes when inflated. The inflatable portion on the tip portion may be used to center the tip portion during a procedure, for example. In some instances, the inflatable portion on the tip portion may be used to protect biological tissue during a procedure, for example.

In an alternative embodiment, a steerable catheter is provided and includes an elongated shaft with a proximal end portion and a distal end portion, an end effector disposed at the distal end portion of the shaft, and a handle disposed at the proximal end portion of the shaft. The end effector includes a proximal end portion disposed proximate the distal end portion of the shaft, and a distal tip portion disposed distally on the end effector. The handle includes a steering actuator rotatably disposed on the handle. The tip portion is movable relative to the shaft. The proximal end portion of the end effector is movable relative to the shaft. The steering actuator enables a user to steer the tip portion and the proximal end portion of the end effector by moving the tip portion and the proximal end portion of the end effector with the steering actuator.

In some embodiments, the diameter of the tip portion may be equal to the diameter of the end effector. In some embodiments the diameter of the end effector may be greater than the diameter of the tip portion. The diameter of the shaft may be approximately equal the end effector diameter. The tip portion may be steerable relative to the shaft in a tip portion steering plane. The handle may include a tip portion steering actuator disposed on the handle. The steerable catheter may include a first tip portion steering line operatively connecting the tip portion and the tip portion steering actuator enabling a user to steer the tip portion in a first direction in the tip portion steering plane. The tip portion may include a steering ring disposed on the tip portion. The first tip portion steering line may operatively connect the tip portion steering ring and the tip portion steering actuator enabling a user to steer the tip portion in a first direction in the tip portion steering plane. The steerable catheter may include a second tip portion steering line operatively connecting the tip portion and the tip portion steering actuator enabling a user to steer the tip portion in a second direction, the second direction being generally opposite the first direction in the tip portion steering plane. The second tip portion steering line may operatively connect the tip portion steering ring and the tip portion steering actuator enabling a user to steer the tip portion in the second direction. In alternative or additional aspects, the end effector may be steerable relative to the shaft in an end effector steering plane. The handle may include an end effector steering actuator disposed on the handle. The steerable catheter may include a first end effector steering line operatively connecting the end effector and the end effector steering actuator enabling a user to steer the end effector in a first direction in the end effector steering plane. The end effector may include an end effector steering ring disposed on the end effector. The first end effector steering line may operatively connect the end effector steering ring and the end effector steering actuator enabling a user to steer the end effector in the first direction in the end effector steering plane. The steerable catheter may include a second end effector steering line operatively connecting the end effector and the end effector steering actuator enabling a user to steer the end effector in a second direction, the second direction being generally opposite the first direction in the end effector steering plane. The steerable catheter may include a second end effector steering line operatively connecting the end effector steering ring and the end effector steering actuator enabling a user to steer the end effector in the second direction. In some embodiments, the tip portion may be steerable relative to the shaft in a tip portion steering plane, and the end effector may be steerable relative to the shaft in an end effector steering plane. The tip portion steering plane and the end effector steering plane may be substantially parallel. The end effector steering plane may be disposed at a steering plane angle relative to the tip portion steering plane, the steering plane angle being greater than zero. In some embodiments the steering plane angle may be about 90 degrees. The end effector steering plane may be substantially perpendicular to the tip portion steering plane. It is understood that the steerable catheter may include any element or elements, method or methods, or combination thereof as disclosed herein.

A method of creating an opening in a biological tissue membrane is provided. The method may include advancing an end effector of a dilator to proximate a biological tissue membrane, creating an initial opening through the membrane by advancing a tip portion of the end effector through the membrane, expanding the initial opening to an expanded opening by advancing at least a portion of a dilation portion of the end effector through the membrane, and withdrawing the end effector from the expanded opening. In some embodiments, creating the initial opening may include energizing a tip portion energizing element of the tip portion. Energizing the tip portion energizing element may include supplying at least one of radio frequency energy and electrocautery energy to the tip portion energizing element. Energizing the tip portion energizing element may include energizing the tip portion energizing element before advancing the tip portion through the membrane. The method of energizing the tip portion energizing element may include energizing the tip portion energizing element while advancing the tip portion through the membrane. Advancing the end effector of the dilator to proximate the biological tissue membrane may include advancing the dilator on a guidewire extending through a central lumen of the dilator.

In some embodiments, expanding the initial opening may include energizing a dilation portion energizing element of the dilation portion. Energizing the dilation portion energizing element may include supplying at least one of radio frequency energy and electrocautery energy to the dilation portion energizing element. Energizing the dilation portion energizing element may include energizing the dilation portion energizing element before advancing at least the portion of the dilation portion through the membrane. Energizing the dilation portion energizing element may include energizing the dilation portion energizing element while advancing at least the portion of the dilation portion through the membrane. Energizing the dilation portion energizing element may include energizing the dilation portion energizing element after advancing at least the portion of the dilation portion through the membrane.

In alternative or additional aspects, the method of creating an opening in a biological tissue membrane may include at least partially expanding an expandable element disposed on the dilation portion of the end effector. At least partially expanding the expandable element may include at least partially inflating an inflatable element. At least partially inflating the inflatable element may include injecting a fluid into the inflatable element. The method may include advancing the at least partially expanded expandable element through the membrane. The method may include withdrawing the at least partially expanded expandable element through the membrane. Expanding the expandable element may include expanding the expandable element while the expandable element is at least partially in contact with the membrane.

In some methods, advancing the end effector may include steering the tip portion in a tip portion steering plane. Advancing the end effector may include steering the dilation portion in a dilation portion steering plane. The tip portion steering plane may be substantially parallel with the dilation portion steering plane. In alternative embodiments the tip portion steering plane may be disposed at a steering plane angle relative to the dilation portion steering plane, the steering plane angle being greater than zero.

In alternative or additional aspects, the biological tissue membrane may include a lamina terminalis and advancing the end effector of the dilator to the biological tissue membrane may include advancing the end effector of the dilator to the lamina terminalis through one of a lumbar puncture and a C1-C2 puncture. The method may include at least partially expanding an expandable element disposed on the tip portion of the end effector. At least partially expanding the expandable element may include at least partially inflating an inflatable element. At least partially inflating the inflatable element may include injecting a fluid into the inflatable element. The method may include advancing the at least partially expanded expandable element through the membrane. The method may include withdrawing the at least partially expanded expandable element through the membrane. Expanding the expandable element may include expanding the expandable element while the expandable element is at least partially in contact with the membrane.

In some embodiments, the steerable catheter or dilator may include a stent portion disposed on the end effector. The stent portion may be used in some procedures. The stent portion may be generally cylindrical. The stent portion may be generally flexible. The stent portion may be generally tapered at the distal end of the stent and the stent portion may have a stent diameter and distal end diameter. The stent diameter may be greater than the tip portion diameter and the dilation end diameter may be less than the tip portion diameter which may allow the stent portion to be directed to a location by a user using the dilator. The stent portion may be disposed on the dilation portion of the end effector. The dilator may be withdrawn from the stent portion leaving the stent portion in the location. A method of using a dilator may include advancing an end effector, with a stent portion installed on the end effector, to a location and withdrawing the end effector from the stent portion, leaving the stent in the location.

In alternative embodiments, the steerable catheter or dilator may include a light element disposed on the tip portion, and the light element may be configured to be operatively connected to a source of light. The source of light may be configured to deliver light to the light element. The source of light may produce red and near infra-red light for medical treatment, for example. Other types of light may be used as well. The light may originate from a laser or a light emitting diode (LED), and may be pulsed and/or continuous, for example. It should be understood that other sources of light may be used. The catheter may include a fiber optic harness extending from the light element, through the shaft, and through the handle. The steerable catheter may be part of a catheter system, the system including the catheter (such as a dilator, or more generally another catheter) and a light generator, the light generator comprising the source of light.

A method of applying light to the brain and/or spinal cord is provided, the method including advancing an end effector of a catheter to proximate a biological tissue, and applying light to the biological tissue. Applying light to the biological tissue may include energizing a light element of the tip portion. Energizing the light element may include supplying light to the light element. The source of light may produce red and near infra-red light for medical treatment, for example. Other types of light may be used as well. The light may originate from a laser or a light emitting diode (LED), and may be pulsed and/or continuous, for example. It should be understood that other sources of light may be used.

Additional features, options or aspects of the invention will become more apparent through a review of a detailed description of various illustrative embodiments described in more detail herein, taken in conjunction with the accompanying drawings of these illustrative embodiments.

### BRIEF DESCRIPTION OF DRAWINGS

FIG. 1 is an isometric view of an illustrative steerable dilator.

FIG. 2 is an isometric view of an illustrative steerable dilator end effector shown in FIG. 1.

FIG. 3 is a sectional view of an exemplary steerable dilator shaft.

FIG. 4 is a sectional view of an alternate exemplary steerable dilator shaft.

FIG. 5 is a sectional view of an illustrative steerable dilator end effector.

FIG. 6 is an alternate sectional view of the illustrative steerable dilator end effector shown in FIG. 5.

FIG. 7 is a detailed isometric view of a portion of an illustrative steerable dilator handle shown in FIG. 1.

FIG. 8 is a detailed isometric view of an illustrative actuator mechanism of the handle in FIG. 7.

FIG. 9 is a sectional view of a portion of the handle shown in FIG. 7.

FIG. 10 is a detailed isometric view of the proximal end portion of the handle shown in FIG. 1.

FIG. 11 is a simplified electrical schematic of an illustrative circuit for delivering radio frequency energy.

FIG. 12 is a simplified electrical schematic of an illustrative circuit for delivering electrocautery power.

FIG. 13 is an isometric view of an alternative illustrative steerable dilator.

FIG. 14 is an isometric view of an alternative illustrative steerable dilator end effector shown in FIG. 13.

FIG. 15 is an isometric cutaway view of an alternative illustrative steerable dilator handle shown in FIG. 13.

FIG. 16 is a sagittal computed tomography image of a patient's head.

FIG. 17 is an isometric view of an exemplary membrane puncture procedure utilizing a steerable dilator.

FIG. 17A is an isometric view of an exemplary membrane puncture procedure utilizing an alternate steerable dilator.

FIG. 18 is another isometric view of an exemplary membrane puncture procedure utilizing the steerable dilator of FIG. 17.

FIG. 18A is another isometric view of an exemplary membrane puncture procedure utilizing the steerable dilator of FIG. 17A.

FIG. 19 is another isometric view of an exemplary membrane puncture procedure utilizing the steerable dilator of FIG. 17.

FIG. 19A is another isometric view of an exemplary membrane puncture procedure utilizing the steerable dilator of FIG. 17A.

FIG. 20 is an isometric view of an alternate illustrative steerable dilator.

FIG. 21 is an isometric view of an alternate illustrative steerable dilator end effector shown in FIG. 20.

FIG. 22 is another isometric view of the alternate illustrative steerable dilator end effector of FIG. 21.

FIG. 23A is a sectional view of the alternate illustrative steerable dilator end effector of FIG. 21.

FIG. 23B is a sectional view of an alternate illustrative steerable dilator end effector.

FIG. 24A is a sectional view of the alternate illustrative steerable dilator dilation portion of FIG. 23A.

FIG. 24B is a sectional view of the alternate illustrative steerable dilator dilation portion of FIG. 23B.

FIG. 25 is another sectional view of the alternate illustrative steerable dilator dilation portion of FIG. 22.

FIG. 26 is a sectional view of the alternate illustrative steerable dilator tip portion of FIG. 22.

FIG. 27 is another sectional view of the alternate illustrative steerable dilator tip portion of FIG. 26.

FIG. 28 is another sectional view of the alternate illustrative steerable dilator tip portion of FIG. 26.

FIG. 29 is an isometric sectional view of the alternate illustrative steerable dilator handle shown in FIG. 20.

FIG. 30 is a detailed isometric cutaway view of the illustrative steerable dilator handle shown in FIGS. 20 and 29.

FIG. 31 is a detailed isometric view of the alternate illustrative steerable dilator steering actuator of FIG. 30.

FIG. 32 is an exploded view of the alternate illustrative steerable dilator steering actuator shown in FIG. 31.

FIG. 33 is a sectional view of the alternate illustrative steerable dilator handle, shaft, and steering actuator of FIG. 20.

FIG. 34 is a sectional view of an alternate illustrative steerable dilator end effector and shaft of FIG. 21.

FIG. 35 is a sectional view of the alternate illustrative steerable dilator handle, shaft, and steering actuator of FIG. 20.

FIG. 36 is a sectional view of the alternate illustrative steerable dilator end effector and shaft of FIG. 21.

FIG. 37 is a sectional view of the alternate illustrative steerable dilator handle, shaft, and steering actuator of FIG. 20.

FIG. 38 is a sectional view of the alternate illustrative steerable dilator end effector and shaft of FIG. 21.

FIG. 39 is an isometric sectional view of the illustrative handle, shaft, and steering actuator of FIG. 20.

FIG. 40 is an isometric sectional view of the illustrative handle, shaft, and steering actuator of FIG. 20.

FIG. 41 is a sectional view of the alternate illustrative steerable dilator handle, shaft, and steering actuator of FIG. 20.

FIG. 42 is a sectional view of the alternate illustrative steerable dilator handle, shaft, and steering actuator of FIG. 20.

FIG. 43 is a sectional view of the alternate illustrative steerable dilator handle, shaft, and steering actuator of FIG. 20.

FIG. 44 is a sectional view of the alternate illustrative steerable dilator end effector and shaft of FIG. 21.

FIG. 45 is a sectional view of the alternate illustrative steerable dilator end effector and shaft of FIG. 21.

FIG. 46 is a sectional view of the alternate illustrative steerable dilator end effector and shaft of FIG. 21.

FIG. 47 is a sectional view of an illustrative stent disposed on a steerable dilator end effector.

FIG. 48 is another sectional view of the illustrative stent disposed on a steerable dilator end effector of FIG. 47.

FIG. 49, is another sectional view of the illustrative stent disposed on a steerable dilator end effector of FIG. 47.

FIG. 50 is a sectional view of the illustrative stent of FIG. 247 in position after a procedure.

FIG. 51 is a sectional view of an alternate illustrative steerable dilator.

FIG. 52 is a sectional view of an alternate illustrative steerable dilator end effector shown in FIG. 51.

### DETAILED DESCRIPTION

Illustrative embodiments according to at least some aspects of the present disclosure are described and illustrated below and include devices and methods relating to surgical procedures. Of course, it will be apparent to those of ordinary skill in the art that the embodiments discussed below are examples and may be reconfigured without departing from the scope and spirit of the present disclosure. It is also to be understood that variations of the exemplary embodiments contemplated by one of ordinary skill in the art shall concurrently comprise part of the instant disclosure. However, for clarity and precision, the illustrative embodiments as discussed below may include optional steps, methods, and features that one of ordinary skill should recognize as not being a requisite to fall within the scope of the present disclosure.

The present disclosure includes, among other things, steerable dilators. As used herein, the term steerable dilator, or simply dilator, includes steerable catheters with a dilation mechanism. It should be understood that some embodiments described herein may be used with steerable catheters without a dilation mechanism. Some illustrative embodiments according to at least some aspects of the present disclosure may be used in connection with creating openings in tissues during minimally invasive neurosurgical procedures. For example, illustrative apparatus and methods disclosed herein may be utilized in connection with minimally invasive surgical treatment of hydrocephalus involving creation of an opening through the lamina terminalis. Some illustrative apparatus and methods disclosed herein may be utilized in connection with PBMT, for example. While the present detailed description of illustrative embodiments focuses on minimally invasive neurosurgical procedures, it will be appreciated that various embodiments according to at least some aspects of the present disclosure may be utilized in connection with other procedures. Additionally, although some features may be described herein in connection with particular exemplary embodiments, one of skill in the art will appreciate that any features described herein may be used alone or in any combination within and between various embodiments.

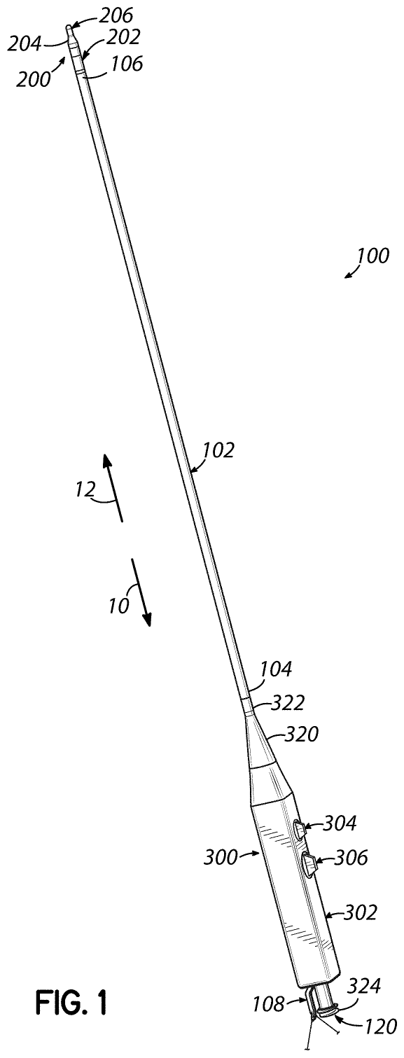

FIG. 1 is an isometric view of an illustrative steerable dilator 100, according to at least some aspects of the present disclosure. The illustrative dilator 100 includes an elongated shaft 102 having a proximal end portion 104 and a distal end portion 106. As used herein to describe various embodiments from the perspective of a user of a surgical device, “proximal” may refer to a direction generally towards the user of the device, while “distal” may refer to a direction generally away from the user of the device. Similarly, in the context of a surgical device inserted into a patient's body and from the perspective of a user of the device, “proximal” may refer to a direction generally away from the center of the patient's body, and “distal” may refer to a direction generally towards the center of the patient's body. For reference, arrow 10 points generally proximally and arrow 12 points generally distally. The illustrative dilator 100 includes an end effector 200 disposed at the distal end portion 106 of the shaft 102, a handle 300 disposed at the proximal end portion 104 of the shaft 102, and a wiring harness 108 configured to operatively connect a source of electrical energy (e.g., an electrosurgical generator) with the end effector 200.

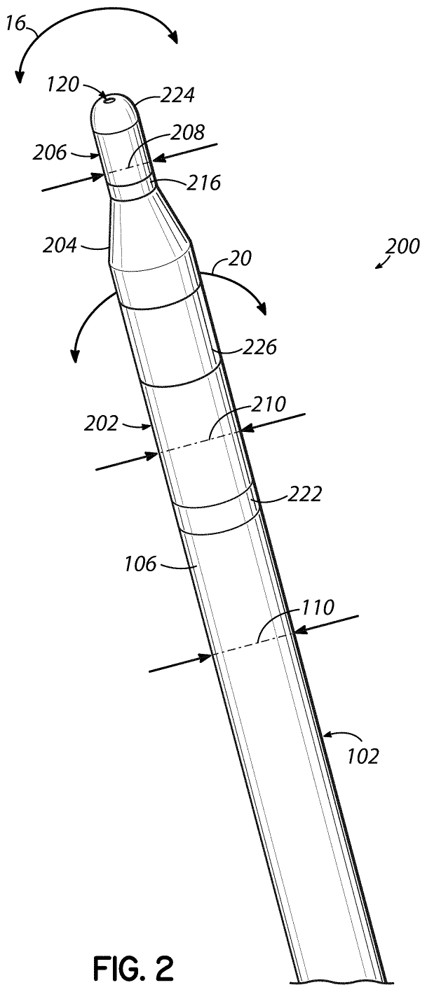

FIG. 2 is an isometric view of an illustrative end effector 200, according to at least some aspects of the present disclosure. The illustrative end effector 200 includes a proximal dilation portion 202 disposed proximate the distal end portion 106 of the shaft 102, a transition portion 204 disposed distally on the dilation portion 202, and a distal tip portion 206 disposed distally on the transition portion 204. The tip portion 206 has a tip portion diameter 208 and the dilation portion has a dilation portion diameter 210. The dilation portion diameter 210 is greater than the tip portion diameter 208. The transition portion 204 tapers generally smoothly (e.g., generally linearly) from the tip portion diameter 208 to the dilation portion diameter 210 between the tip portion 206 and the dilation portion 202. In some embodiments, a shaft diameter 110 may approximately equal the dilation portion diameter 210.

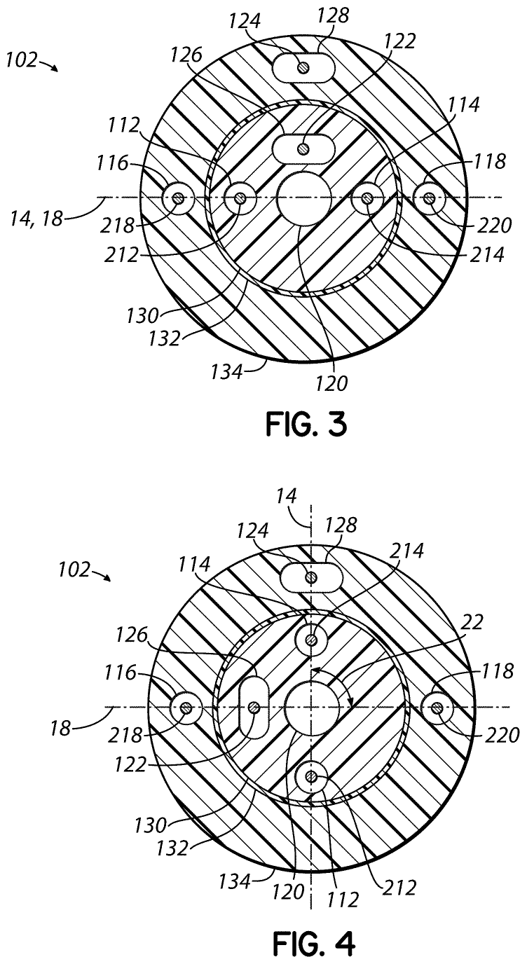

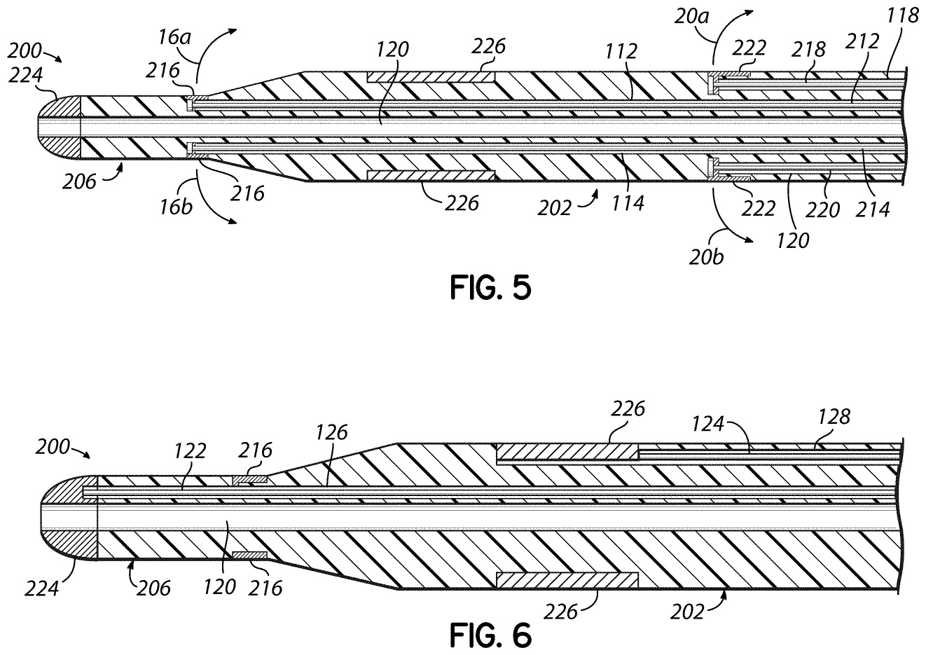

FIGS. 3 and 4 are sectional views of exemplary configurations of the shaft 102 and FIG. 5 is a sectional view of the illustrative end effector 200, all according to at least some aspects of the present disclosure. Referring to FIGS. 2-5, the illustrative end effector 200 is configured so that the tip portion 206 is steerable in a tip portion steering plane 14 as shown by arrow 16 and the dilation portion 202 is steerable in a dilation portion steering plane 18 as shown by arrow 20. Generally, the tip portion 206 is steerable relative to the dilation portion 202 and the dilation portion 202 is steerable relative to the shaft 102. As another option, only one of the tip portion 206 or the dilation portion 202 may be steerable.

In some embodiments, the tip portion steering plane 14 and the dilation portion steering plane 18 are substantially parallel, such as substantially coplanar. Such an embodiment is shown in FIGS. 3 and 5. In some embodiments, the dilation portion steering plane 18 is disposed transversely relative to the tip portion steering plane 14. As used herein, “transverse” may refer to relative angular orientations that are non-parallel (e.g., perpendicular or oblique). For example, the dilation portion steering plane 18 may be disposed at a steering plane angle 22 of about 90 degrees relative to the tip portion steering plane 14 as shown in FIG. 4. Such a configuration may be advantageous for some uses in which it may be desirable to utilize 3D steering of the dilator 100 relative to the longitudinal axis of the shaft 102. In other embodiments, the steering plane angle 22 may be greater than about 0 to about 90 degrees, for example. The common components of the embodiment of FIGS. 3 and 5 and the embodiment of FIG. 4 are described together with like reference numbers, however, it will be understood that the relative orientations of some components may differ in various exemplary embodiments.

Referring to FIGS. 2-5, the illustrative end effector 200 is steerable through the action of one or more tension elements on one or more steering rings. The tip portion 206 is steerable in the tip portion steering plane 14 by the selective distal pulling of a first tip portion steering line 212 and a second tip portion steering line 214 on a tip portion steering ring 216. Specifically, pulling distally on the first tip portion steering line 212 pulls on the tip portion steering ring 216 so as to cause the tip portion 206 to flex in the direction of arrow 16a. Pulling distally on the second tip portion steering line 214 pulls on the tip portion steering ring 216 so as to cause the tip portion 206 to flex in the direction of arrow 16b. The directions of arrow 16a and the direction of arrow 16b may be generally opposite directions. The tip portion steering lines 212, 214 may be connected to the tip portion steering ring 216 by a weld or solder joint, or a mechanical fastener, for example. In some embodiments, the tip portion steering ring 216 may be constructed of a radiopaque material, which may facilitate visualization of the dilator 100 using various medical imaging modalities.

Similarly, the dilation portion 202 is steerable in the dilation portion steering plane 18 by the selective distal pulling of a first dilation portion steering line 218 and a second dilation portion steering line 220 on a dilation portion steering ring 222. Specifically, pulling distally on the first dilation portion steering line 218 pulls on the dilation portion steering ring 222 so as to cause the dilation portion 202 to flex in the direction of arrow 20a. Pulling distally on the second dilation portion steering line 220 pulls on the dilation portion steering ring 222 so as to cause the dilation portion 202 to flex in the direction of arrow 20b. The direction of arrow 20a and the direction of arrow 20b may be generally opposite directions. The dilation portion steering lines 218, 220 may be connected to the dilation portion steering ring 222 by a weld or solder joint, or a mechanical fastener, for example. In some embodiments, the dilation portion steering ring 222 may be constructed of a radiopaque material, which may facilitate visualization of the dilator 100 using various medical imaging modalities.

As described below, the tension elements (e.g., steering lines 212, 214, 218, 220) are tensioned by one or more actuators disposed on the handle 300. Generally, the tension elements (e.g., steering lines 212, 214, 218, 220) may be constructed of any materials capable of transmitting a tensile force from the actuators to the steering rings 216, 222. For example, the steering lines 212, 214, 218, 220 may comprise metal wires and/or various suture materials.

FIG. 6 is a sectional view of the illustrative end effector 200, according to at least some aspects of the present disclosure. Referring to FIGS. 1, 2, and 6, the illustrative end effector 200 includes one or more energizing elements, such as a tip energizing element 224, which forms the distal end of the tip portion 206, and a dilation energizing element 226, which forms a circumferential surface of a portion of the dilation portion 202. As described below, the energizing elements 224, 226 of the end effector 200 are configured to receive electrical energy via the wiring harness 108 and are configured to deliver the electrical energy to biological tissues. The illustrative wiring harness 108 includes a tip portion power wire 122 extending through the shaft 102 to the tip energizing element 224 and a dilation portion power wire 124 extending through the shaft 102 to the dilation energizing element 226. Depending on the electrosurgical technique that is employed, each power wire 122, 124 may comprise one or more separate and/or insulated conductors. The power wires 122, 124 are connected to the energizing elements 224, 226 by solder connections, for example. In some embodiments, the energizing elements 224, 226 may be constructed of radiopaque materials, which may facilitate visualization of the dilator 100 using various medical imaging modalities.

In the illustrative embodiment, the distal end of the tip portion 206 is defined at least partially by the tip energizing element 224. The tip energizing element 224 is shaped to facilitate passage of the tip portion 206 through a biological tissue membrane. The tip energizing element 224 may include a radiused shape (e.g., generally rounded) as shown and/or other shapes, such as bevels and/or tapers (e.g., generally pointed).

Referring to FIGS. 1, 3, and 4, the shaft 102 may include one or more longitudinal lumens extending from the proximal end portion 104 to the distal end portion 106. In the illustrative embodiment, each of the steering lines 212, 214, 218, 220 is slidably disposed within a respective steering line lumen 112, 114, 116, 118. In this exemplary embodiment, the lumens of each respective pair of steering line lumens 112, 114, 116, 118 are positioned substantially diametrically opposite each other (e.g., about 180 degrees relative to each other). Alternative embodiments may comprise only a single steering line 212, 214, 218, 220 and/or steering line lumen 112, 114, 116, 118 for a particular steerable portion 202, 206, which may provide unidirectional steering for that portion 202, 206.

In this illustrative embodiment, a guidewire lumen 120 extends longitudinally through the end effector 200, shaft 102, and handle 300, and is configured to slidably receive a guidewire therethrough. In the illustrative embodiment, the guidewire lumen 120 is substantially centrally located in the shaft 102 and the end effector 200. The tip portion power wire 122 extends through a tip portion power wire lumen 126 and the dilation portion power wire 124 extends through a dilation portion power wire lumen 128.

The shaft 102 may be constructed from one or more layers. For example, this illustrative embodiment includes an inner layer 130, an intermediate layer 132, and an outer layer 134. In some embodiments, the layers 130, 132, 134 are formed separately and are then joined, such as using a thermal bonding process and/or a chemical bonding process. Alternatively, two or more of the layers 130, 132, 134 may be formed together.

In this illustrative embodiment, the inner layer 130 comprises a polymer, such as a polymer extrusion. The inner layer 130 may be substantially uniform over the length of the shaft 102, or some characteristics may vary over the length of the shaft 102. For example, in the illustrative embodiment, the inner layer 130 comprises a plurality of extruded segments facilitating a differing stiffness over the length of the shaft 102. As used herein, “stiffness” may refer to the resistance of an object to deformation under an applied force. In this exemplary embodiment, the stiffness of the inner layer 130 decreases from proximal to distal so that the shaft 102 is generally stiff proximally for pushability (e.g., relatively high buckling strength) and/or is generally softer distally for flexibility and atraumatic contact with tissue. In the illustrative embodiment, the inner layer 130 extends substantially from the handle 300 to the tip portion 206 of the end effector 200. The stiffness changes may occur in discrete intervals. The distal portion (e.g., near the tip portion 206) may comprise a polymer having a stiffness of about Shore 80A or less. The proximal portion (e.g., near the handle 300) may comprise a polymer having a stiffness of about Shore 75D or higher, which may approximately match the stiffness of the handle 300 material. In some embodiments, various intermediate segments (e.g., between a distal-most segment and a proximal-most segment) may vary in stiffness, such as in a stepwise fashion. For example, some intermediate segments may increase in Shore 10D to Shore 15D increments (e.g., 25D, 40D, 55D, 65D) from distal to proximal. In some embodiments, the outer diameter of the inner layer 130 may be about 0.7 mm to about 1.7 mm. Different diameters may be useful when different anatomy will be traversed and/or to accommodate different sized guidewires, for example.

In this illustrative embodiment, the intermediate layer 132 comprises a structural reinforcing layer, such as a polymer (e.g., Nylon, Kevlar) or metal (e.g., stainless steel, nickel titanium) coil and/or braided structure. In the illustrative embodiment, the intermediate layer 132 is configured to resist kinking of the shaft 102 and/or to increase the torsional strength of the shaft 102. In some embodiments, some characteristics of the intermediate layer 132 may vary over the length of the shaft 102. For example, the pitch and/or pick rate of a coil and/or braided intermediate layer 132 may vary over the length of the shaft 102. In one exemplary embodiment, the braid and/or coil may be more open (providing better pushability due to the pattern extending more axially) toward the proximal end portion 104 of the shaft 102 and/or the braid and/or coil may be more closed (providing better flexibility due to the pattern being positioned more radially) toward the distal end portion 106 of the shaft 102. Various shapes of filaments and/or wires may be utilized. For example, a filament or wire may have a generally round cross section, or it may have a generally rectangular cross section, such as in devices requiring a smaller tip portion diameter 208. Some illustrative embodiments may not include a structural reinforcing layer (e.g., intermediate layer 132). For example, a device configured for use where the pathway from the access site to the treatment site is not tortuous may not require the torsional stiffness and/or kink resistance provided by such an intermediate later 132. In the illustrative embodiment, the intermediate layer 132 extends substantially from the proximal side of the tapered transition portion 204 to the distal side of the handle 300.

In this illustrative embodiment, the outer layer 134 comprises a polymer, such as a polymer extrusion. The outer layer 134 may be substantially uniform over the length of the shaft 102, or some characteristics may vary over the length of the shaft 102. For example, in the illustrative embodiment, the outer layer 134 comprises a plurality of extruded segments facilitating a differing stiffness over the length of the shaft 102. In this exemplary embodiment, the stiffness of the outer layer 134 decreases from proximal to distal so that the shaft 102 is generally stiff proximally for pushability (e.g., relatively high buckling strength) and/or is generally softer distally for flexibility and atraumatic contact with tissue. The outer layer 134 extends substantially from the distal side of the handle 300 to the tapered transition portion 204 of the end effector 200. In this illustrative embodiment, the outer layer 134 is thermally formed to at least partially define the tapered transition portion 204. The stiffness changes may occur in discrete intervals. The dilation portion 202 of the end effector may comprise a polymer having a stiffness of about Shore 25D or less. The proximal portion (e.g., near the handle 300) may comprise a polymer having a stiffness of about Shore 75D or higher. In some embodiments, various intermediate segments (e.g., between a distal-most segment and a proximal-most segment) may vary in stiffness, such as in a stepwise fashion. For example, some intermediate segments may increase in Shore 10D to Shore 15D increments (e.g., 40D, 55D, 65D) from distal to proximal. In some embodiments, the segments of the inner layer 130 may be positioned to overlap the segments of the outer layer 134, which may provide generally smoother transitions over the length of the shaft 102. In some embodiments, the outer diameter of the outer layer 134 may be about 2.3 mm to about 3.0 mm. Different diameters may be useful for creating different opening sizes at the treatment site, for example.

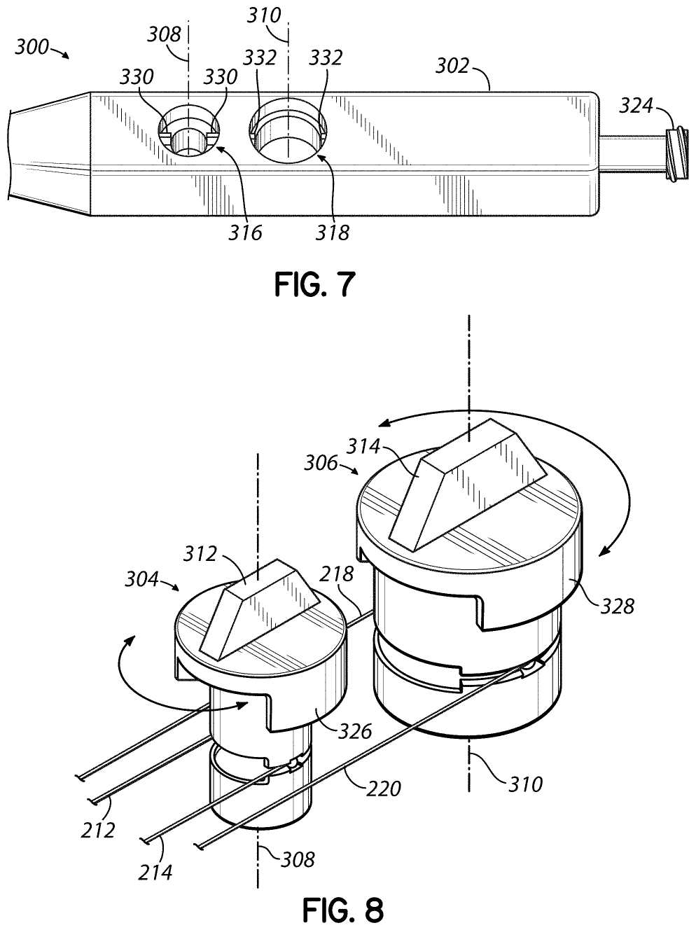

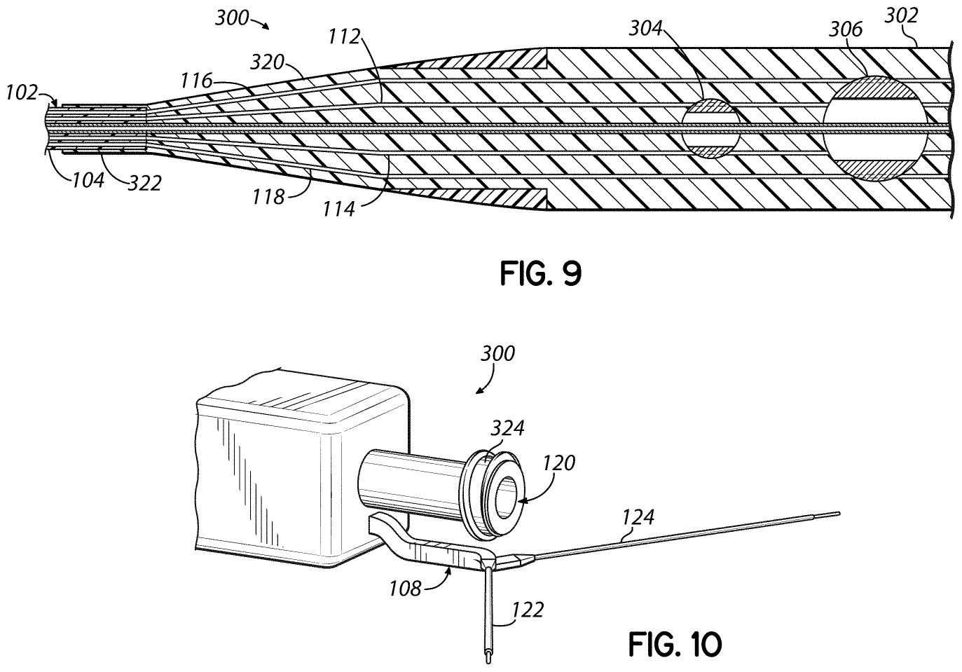

FIG. 7 is a detailed isometric view of a portion of an illustrative handle 300, FIG. 8 is a detailed isometric view of an illustrative actuator mechanism of the handle 300, FIG. 9 is a cutaway view of a portion of the handle 300, and FIG. 10 is a detailed isometric view of the proximal end portion of the handle 300, all according to at least some aspects of the present disclosure. Referring to FIGS. 1 and 7, the illustrative handle 300 comprises a handle body 302 generally configured to be gripped by a user's hand. The handle body 302 may be constructed of a generally rigid polymer (e.g., Shore 75D or higher).

Referring to FIGS. 1, 5, and 7-9, the illustrative handle 300 includes a tip portion steering actuator 304 and a dilation portion steering actuator 306. In this exemplary embodiment, each steering actuator 304, 306 is generally in the form of a knob that is rotatably disposed on the handle 300 for operation by the user. More specifically, in this embodiment, each steering actuator 304, 306 is rotatable about a respective axis 308, 310 that is substantially normal to the portion of the handle 300 where the respective steering actuator 304, 306 is exposed. Each steering actuator 304, 306 comprises a grip element, such as an upstanding tab 312, 314. The steering actuators 304, 306 are rotatably mounted on the handle 300 in respective recesses 316, 318. In some embodiments, the steering actuators 304, 306 and/or the recesses 316, 318 may be provided with engagement features (e.g., rings) facilitating a snap fit of the steering actuators 304, 306 into the respective recesses 316, 318. It will be appreciated that alternative steering actuators, such as sliders or rollers, may be configured to operate in a generally similar manner.

The tip portion steering actuator 304 is coupled to the first tip portion steering line 212 and the second tip portion steering line 214. In this illustrative embodiment, the tip portion steering lines 212, 214 are coupled to the tip steering actuator 304 such that rotating the tip portion steering actuator 304 clockwise is operative to tension the first tip portion steering line 212. Likewise, rotating the tip steering actuator 304 counter-clockwise is operative to tension the second tip portion steering line 214. Similarly, the dilation portion steering actuator 306 is coupled to the first dilation portion steering line 218 and the second dilation portion steering line 220. The dilation portion steering lines 218, 220 are coupled to the dilation portion steering actuator 306 such that rotating the dilation portion steering actuator 306 clockwise is operative to tension the first dilation portion steering line 218. Likewise, rotating the dilation portion steering actuator 306 counter-clockwise is operative to tension the second dilation portion steering line 220. Generally, in this illustrative embodiment, the respective diameters of the steering actuators 304, 306 where the steering lines 212, 214, 218, 220 are attached determine the degree of “pull” on the respective steering lines 212, 214, 218, 220 and, thus, the resultant deflection of the tip portion 206 and/or the dilation portion 202.

In this illustrative embodiment, the tip portion steering lines 212, 214 are individual, discrete lengths that are individually attached to opposite, lateral sides of the tip portion steering actuator 304. In alternative embodiments, both tip portion steering lines 212, 214 may be provided as part of a continuous length that extends from the tip portion steering ring 216, around the tip portion steering actuator 304, and back to the tip portion steering ring 216. In some such embodiments, the tip portion steering lines 212, 214 may be secured to the tip portion steering actuator 304 at one location, such as on a proximal aspect of the tip portion steering actuator 304. Generally, the tip portion steering lines 212, 214 may be secured to the tip portion steering actuator 304 by the engagement of one or more raised features on the tip portion steering lines 212, 214 with a corresponding recess on the tip portion steering actuator 304. Alternatively, the tip portion steering lines 212, 214 may be welded or soldered to the tip portion steering actuator 304. The dilation portion steering lines 218, 220 and the dilation portion steering actuator 306 may be configured in a similar manner.

Referring to FIGS. 7 and 8, the steering actuators 304, 306 and the recesses 316, 318 may be provided with corresponding rotation limiting features. For example, the steering actuators 304, 306 may include one or more steering limits 326, 328 arranged to selectively contact one or more respective steering stops 330, 332 in the recesses 316, 318 at the desired limits of travel. Generally, it will be appreciated that the degree of steering of the tip portion 206 and/or the dilation portion 202 may depend upon the diameter of the respective steering actuator 304, 306 and/or the degree of angular rotation of the respective steering actuator 304, 306 permitted by the respective rotation limiting features. In some embodiments, one of the tip portion 206 and the dilation portion 202 may be configured to have greater maximum deflection than the other. Alternatively, the tip portion 206 and the dilation portion 202 may be configured to have approximately the same maximum deflection.

Referring to FIGS. 3, 5, and 9, the steering lines 212, 214, 218, 220 are routed through respective steering line lumens 112, 114, 116, 118 from the respective steering actuators 304, 306 to the shaft 102 through a handle transition portion 320, which generally tapers from the handle body 302 to the shaft 102. The steering line lumens 112, 114, 116, 118 are arranged to direct the steering lines 212, 214, 218, 220 at the appropriate spacings to correspond to the diameters of the steering actuators 304, 306. The handle transition portion 320 may include a strain relief portion 322, which may be configured to prevent the shaft 102 from kinking near the handle 300. For example, the strain relief portion 322 may be formed of a polymer with a hardness of about Shore 40D to about Shore 60D that flexes with the proximal end portion 104 of the shaft 102. The steering line lumens 112, 114, 116, 118 continue from the shaft 102 to the respective steering actuators 304, 306 to route the respective steering lines 212, 214, 218, 220.

Referring to FIG. 10, the proximal end portion of the handle 300 includes a proximally extending portion of the wiring harness 108, including power wires 122, 124. Additionally, in this illustrative embodiment, the proximal end portion of the handle 300 includes the proximal end of the guidewire lumen 120. A mechanical coupling 324, such as a female Luer-lock fitting is provided on the guidewire lumen 120 to facilitate, for example, attachment of a syringe for injection of fluid (e.g., saline, contrast) through the guidewire lumen 120. The coupling 324 may be used in connection with insertion of a guidewire and/or to attach a sealing device to prevent fluid leakage via the guidewire lumen 120.

The proximally extending portion of the wiring harness 108 is positioned in the handle 300 to avoid conflicting with the guidewire lumen 120. In some embodiments, the various wires 122, 124 of the wiring harness 108 may be color-coded to correspond to the steering actuators 304, 306, which may reduce the risk of user confusion.

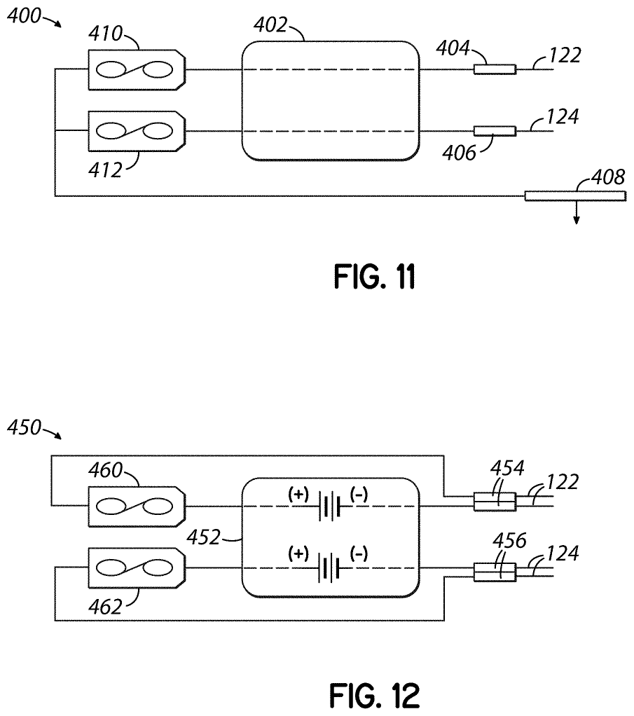

FIG. 11 is a simplified electrical schematic view of an illustrative circuit 400 for delivering radio frequency energy, according to at least some aspects of the present disclosure. In this illustrative embodiment, an electrosurgical generator 402 (e.g., a multi-channel radio frequency (“RF”) generator) is arranged to deliver RF energy to the tip portion power wire 122 and/or the dilation portion power wire 124, which are attached via respective connectors 404, 406. The electrosurgical generator 402 is also coupled to a patient grounding pad 408 via respective switches 410, 412. When the tip portion switch 410 is closed/activated, the electrosurgical generator 402 delivers RF energy to the tip energizing element 224 via the tip portion power wire 122 and the grounding pad 408. Similarly, when the dilation portion switch 412 is closed/activated, the electrosurgical generator 402 delivers RF energy to the dilation energizing element 226 via the dilation portion power wire 124 and the grounding pad 408. In alternative embodiments, the switches 410, 412 may comprise foot pedals, controls on the electrosurgical generator unit 402, or controls disposed on the handle 300, for example.

FIG. 12 is a simplified electrical schematic view of an illustrative circuit 450 for delivering electrocautery power, according to at least some aspects of the present disclosure. In this illustrative embodiment, an electrosurgical generator 452 is arranged to deliver electrocautery energy to the tip portion power wire 122 (e.g., comprising two electrically insulated, separate conductors) and/or the dilation portion power wire 124 (e.g., comprising two electrically insulated, separate conductors), which are attached via respective connectors 454, 456. Respective switches 460, 462 couple the electrosurgical generator 452 to the power wires 122, 124. When the tip portion switch 460 is closed/activated, the electrosurgical generator 452 delivers electrocautery energy to the tip energizing element 224 via the tip portion power wire 122. Similarly, when the dilation portion switch 462 is closed/activated, the electrosurgical generator 452 delivers electrocautery energy to the dilation energizing element 226 via the dilation portion power wire 124. In alternative embodiments, the switches 460, 462 may comprise foot pedals, controls on the electrosurgical generator unit 452, or controls disposed on the handle 300, for example. Separate circuits are used to control power/energy to each energizing element 224, 226 independently.

In various embodiments according to at least some aspects of the present disclosure, various combinations of energizing elements 224, 226 and power circuits 400, 450 may be provided. For example, some embodiments may include no energizing elements 224, 226 and/or no power circuits 400, 450. Other embodiments may include only the RF power circuit 400, which may be configured to supply RF energy to one or both of the tip portion energizing element 224 and/or the dilation portion energizing element 226. Some embodiments may include only the electrocautery power circuit 450, which may be configured to supply electrocautery energy to one or both of the tip portion energizing element 224 and/or the dilation portion energizing element 226. Alternative embodiments may include both the RF power circuit 400 and the electrocautery power circuit 450. In some such embodiments, the RF power circuit 400 may be configured to provide RF energy to only the tip portion energizing element 224 or the dilation portion energizing element 226, and the electrocautery power circuit 450 may be configured to provide electrocautery energy to only the other of the tip portion energizing element 224 and the dilation portion energizing element 226.

RF energy produces a high frequency vibration of the RF element, while electrocautery (EC) energy will produce heat. Therefore, the RF element will remain cooler and essentially act as a dottering instrument. With EC, the tissue is heated and slightly burned. Generally, RF energy causes tissue to also vibrate at high frequency and may cause tissue scarring as a result. RF is typically used for passing an instrument through thin tissue membranes. EC energy is essentially used to stop bleeding during surgery and can also be used to cut and stop bleeding at the same time. Relative to the present disclosure, EC may be used for “fixing” the tissue (where the hole through the tissue is created by the proximal element). Using RF to scar and therefore fix the tissue surrounding the hole may be effective as well.

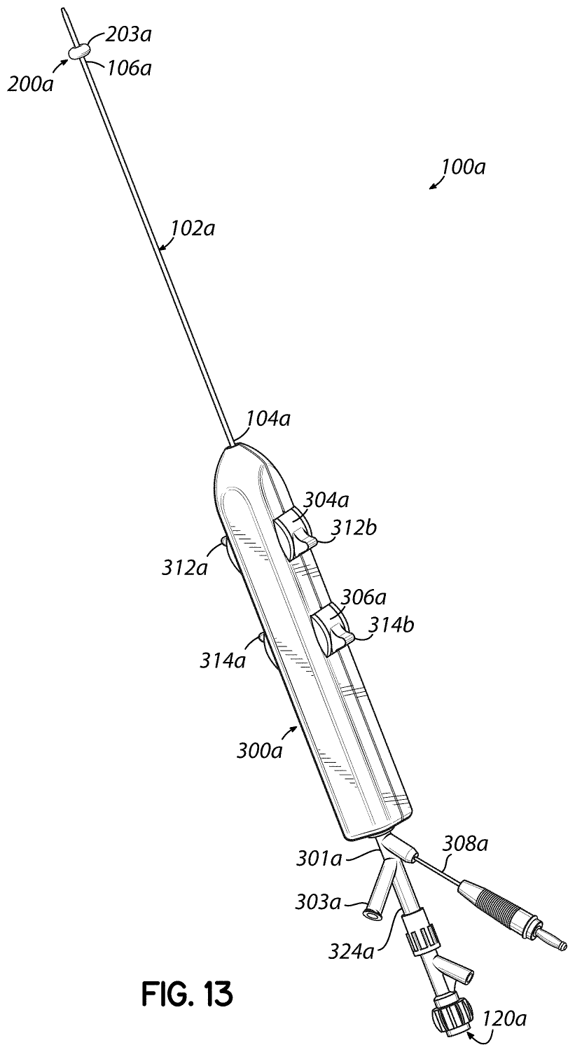

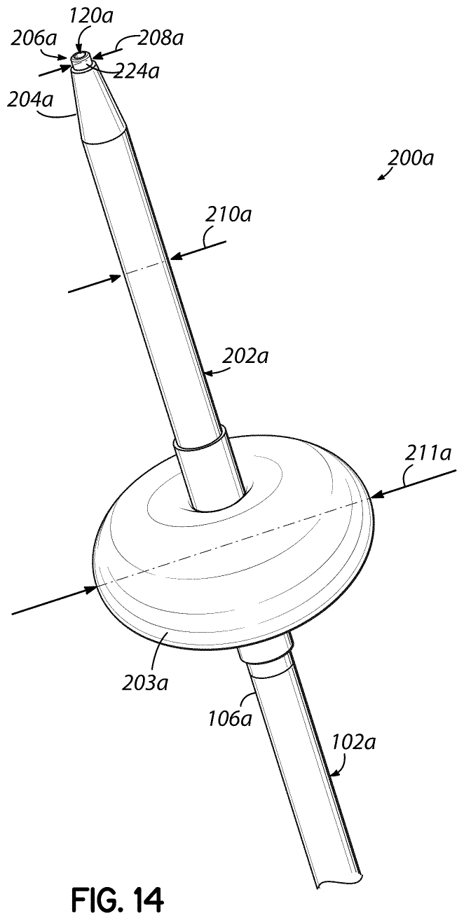

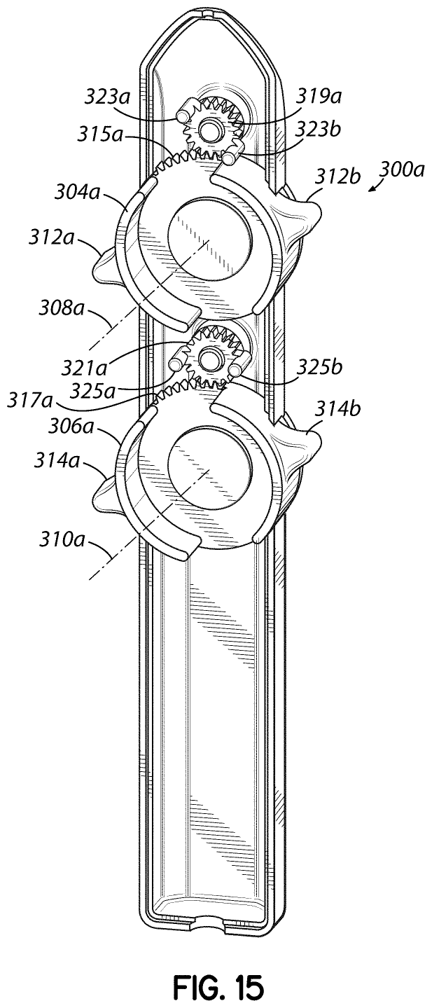

FIG. 13 is an isometric view of an alternative illustrative steerable dilator 100a; FIG. 14 is an isometric view of an alternative illustrative end effector 200a of the dilator 100a; and FIG. 15 is an isometric cutaway view of an alternative illustrative handle 300a of the dilator 100a, all according to at least some aspects of the present disclosure. Generally, the steerable dilator 100a is similar in construction and operation to the steerable dilator 100 described above and any feature(s) of the steerable dilator 100a may be used in various other exemplary embodiments according to the present disclosure. Like reference numbers refer to like components and attendant function. For brevity, the following description omits redundant description and focuses on the differences between the steerable dilator 100a and the steerable dilator 100.

This illustrative dilator 100a includes an elongated shaft 102a having a proximal end portion 104a and a distal end portion 106a. The illustrative dilator 100a includes an end effector 200a disposed at the distal end portion 106a of the shaft 102a and a handle 300a disposed at the proximal end portion 104a of the shaft 102a. The illustrative end effector 200a includes a proximal dilation portion 202a disposed proximate the distal end portion 106a of the shaft 102a, a transition portion 204a disposed distally on the dilation portion 202a, and a distal tip portion 206a disposed distally on the transition portion 204a. The tip portion 206a has a tip portion diameter 208a and the dilation portion 202a has a dilation portion diameter 210a.

In this illustrative embodiment, the dilation portion 202a includes an expandable element 203a, such as an inflatable element, which is selectively expandable from approximately the dilation portion diameter 210a to a fully expanded diameter 211a. The expandable element 203a is configured to be selectively expanded and/or contracted, such as by injecting or withdrawing fluid (e.g., a liquid or a gas) via an inflation connection 303a. The inflation connection 303a is provided on a connection hub 301a at the proximal end of the handle 300a, such as on a dedicated branch of the connection hub 301a. In one exemplary embodiment, the expandable element 203a may comprise a generally toroidal inflatable element configured to have an expanded diameter 211a. A hole is formed in the tissue in a somewhat constricted, non-circular anatomy. For example, the third ventricle is flat like a pancake and only a few mm (3-4 mm max) thick but may be 1-2 cm long or more depending upon the severity of the hydrocephalus. Therefore, the dilation may be more “oval” as opposed to “circular” but could be either shape, or even another shape. A balloon-like expansible element used for dilation should be highly compliant to conform to the anatomy during the procedure. In an uninflated state the balloon may have a diameter of approximately 2 mm. The balloon may be filled with a suitable fluid to inflate and create a fenestration in the tissue of between approximately 2 mm to approximately 10 mm in diameter. In some embodiments, the expandable element 203a comprising an inflatable element may be generally in the form of a compliant balloon having a hardness of about Shore 60A to about Shore 25D. Expandable elements 203a, such as inflatable elements, may be fully inflated or partially expanded, such as by injecting and/or withdrawing fluid to achieve a desired partially or fully expanded diameter, for example.

In this illustrative embodiment, the tip portion 206a comprises a tip portion energizing element 224a and/or the dilation portion 202a may not include an energizing element (e.g., similar to dilation portion energizing element 224 described above). A wiring harness 308a operatively connected to the tip portion energizing element 224a may extend proximally through the connection hub 301a of the handle 300a, such as through a dedicated branch of the connection hub 301a. The tip portion energizing element 224a includes the distal end opening of a guidewire lumen 120a, which extends longitudinally through the handle 300a, shaft 102a, and end effector 200a. The guidewire lumen 120a may be accessed proximally via a mechanical coupling 324a, which may be provided on a dedicated branch of the connection hub 301a.

In this illustrative embodiment, the handle 300a includes a tip portion steering actuator 304a and a dilation portion steering actuator 306a. In this exemplary embodiment, each steering actuator 304a, 306a is generally in the form of a roller that is rotatably disposed on the handle 300a for operation by the user. More specifically, in this embodiment, each steering actuator 304a, 306a is rotatable about a respective axis 308a, 310a that is substantially parallel to the portion of the handle 300 where the respective steering actuator 304a, 306a is exposed. Each steering actuator 304a, 306a is exposed on two opposite faces of the handle 300a. Each steering actuator 304a, 306a comprises at least one grip element, such as upstanding tabs 312a, 312b, 314a, 314b. Each steering actuator 304a, 306a comprises a toothed portion 315a, 317a, which is operatively engaged with a respective toothed gear 319a, 321a. Each toothed gear 319a, 321a comprises at least one post 323a, 323b, 325a, 325b, which is connected to a respective steering line 212, 214, 218, 220. Rotation of a steering actuator 304a, 306a in one direction causes the respective toothed gear 319a, 321a to rotate in the opposite direction. Rotation of the toothed gear 319a, 321a moves the respective posts 323a, 323b, 325a, 325b, thereby tensioning and/or slacking the respective steering lines 212, 214, 218, 220.

Exemplary methods of using an illustrative steerable dilator 100 according to at least some aspects of the present disclosure are described below and may include optional and/or alternative structures and/or operations. Although the following description focuses on the steerable dilator 100 of FIGS. 1-12, it will be appreciated that similar operations are likewise applicable to other exemplary embodiments, including the dilator 100a of FIGS. 13-15.

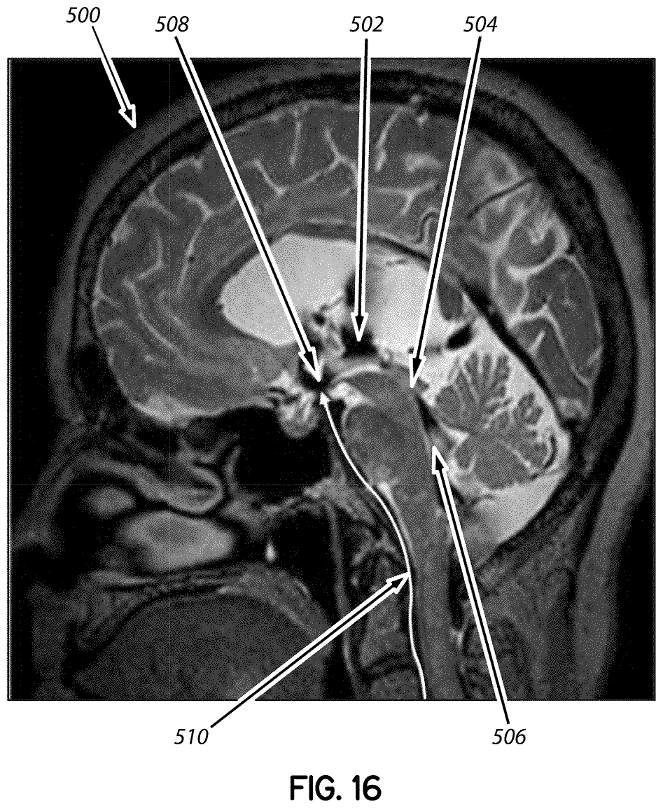

FIG. 16 is a sagittal computed tomography image of a patient's head 500, according to at least some aspects of the present disclosure. In FIG. 16, the third ventricle 502 is distended due to a pressure increase created by a stenosed (narrowed or blocked) aqueduct 504 that prevents CSF from draining naturally from the third ventricle 502 to the fourth ventricle 506, causing a non-communicating hydrocephalus condition. To treat this condition, an exemplary dilator 100 may be used to create an opening through the lamina terminalis 508, which may be accessed by the dilator 100 via the illustrated pathway 510.

Generally, some exemplary methods of creating an opening in a biological tissue membrane (e.g., performing a ventriculostomy) may include accessing the lamina terminalis 508 in a minimally invasive manner, such as via a lumbar puncture or a C1-C2 puncture into the CSF system. Accordingly, the lamina terminalis 508 may be accessed from “below” (e.g., from the central canal), rather than from “above” as in the endoscopic third ventriculostomy procedures described in the Background section.

A surgical device (e.g., dilator 100) may be introduced into the CSF space using a modified Seldinger technique. For example, an initial lumbar or C1-C2 puncture may be made with a hollow needle. Then, a guidewire may be advanced through the lumen of the needle. The needle may be removed and other devices (e.g., dilator 100) may be passed over the guidewire.

Once access to the CSF space is obtained, the dilator 100 may be directed to the desired treatment site (e.g., lamina terminalis 508), such as using fluoroscopic and/or computed tomography imaging techniques. The dilator 100 may be steered as necessary to navigate to the ventriculostomy site and/or to avoid injury to other anatomical structures along the pathway 510 (e.g., blood vessels and/or nerve structures). For example, the tip portion 206 may be steered in the tip portion steering plane 14 and/or the dilation portion 202 may be steered in the dilation portion steering plane 18, such as using steering actuators 304, 306. In some circumstances, it may be advantageous to advance the dilator 100 along a guidewire in a stepwise manner, such as in connection with imaging guidance.

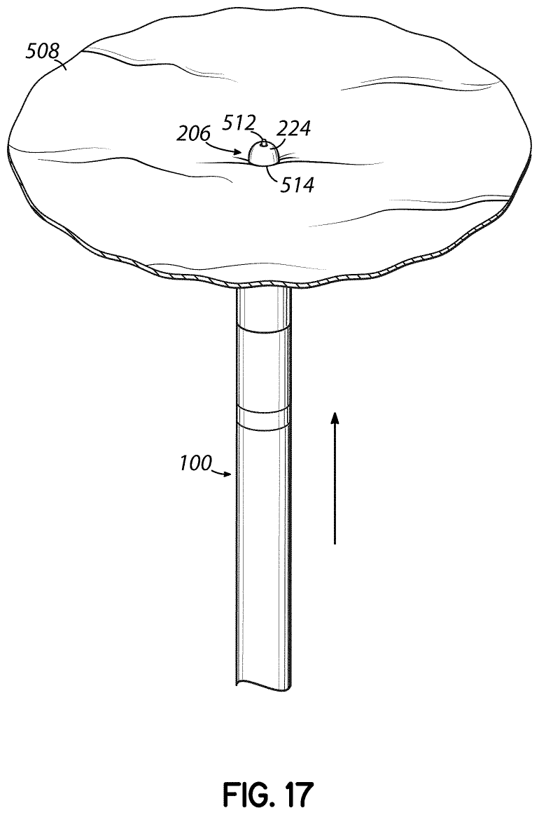

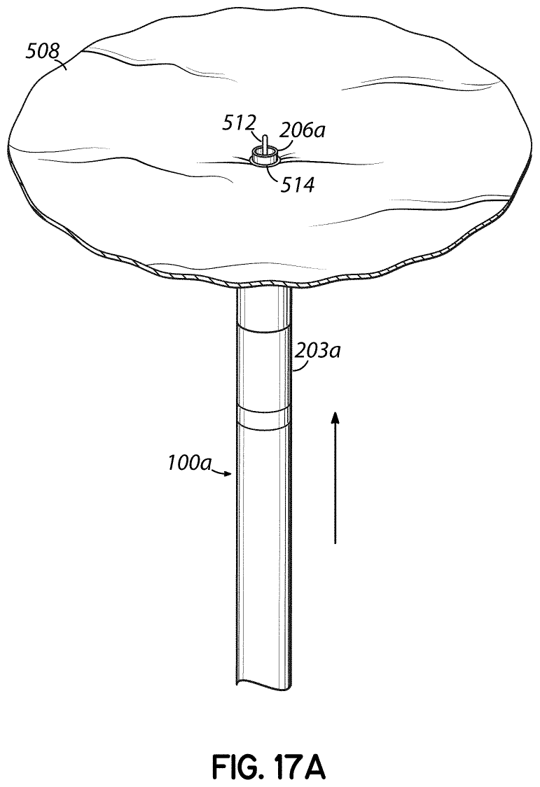

After positioning of the dilator 100 at the desired puncture site is confirmed, such as by “tenting” the lamina terminalis membrane 508, the dilator 100 may be used to create the opening through the membrane 508. FIGS. 17, 18, and 19 are isometric views of an exemplary membrane puncture procedure utilizing the dilator 100, all according to at least some aspects of the present disclosure. Referring to FIG. 17, the dilator 100 may be advanced to mechanically puncture the lamina terminalis 508 with the tip portion 206, creating an initial opening 514. Alternatively, the tip portion energizing element 224 may be energized (e.g., by applying RF and/or electrocautery energy) prior to and/or during advancement of the tip portion 206 through the lamina terminalis 508, which may reduce the tissue deflection while creating the initial opening 514. In some circumstances, the guidewire 512 may be at least partially retracted while creating the initial opening 514. Or, the guidewire 512 may be advanced through the lamina terminalis 508 before the tip portion 206 and/or with the tip portion 206.

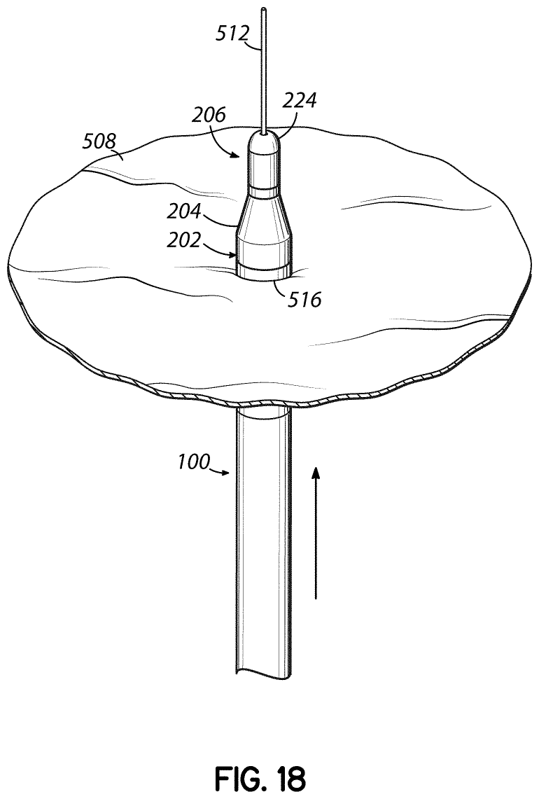

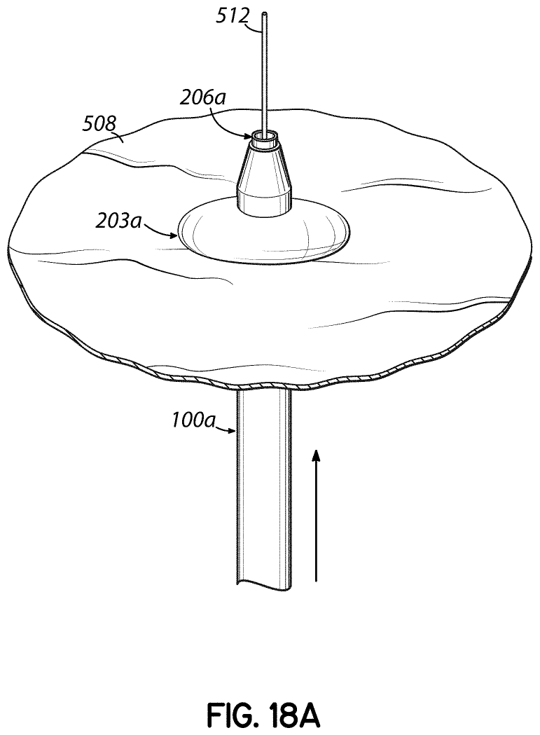

Referring to FIG. 18, after creating the initial opening 514 through the membrane 508, the guidewire 512 may be further advanced beyond the membrane 508. The dilator 100 may be advanced to expand the initial opening 514 to create an expanded opening 516 using the transition portion 204 and/or the dilation portion 202. For example, at least a portion of the dilation portion 202 may be advanced though the membrane 508. The dilator 100 may be advanced to mechanically expand the initial opening 514. Alternatively, the dilation portion energizing element 226 may be energized prior to, during, and/or after advancement of the dilator 100 to treat the expanded opening 516, such as by applying RF and/or electrocautery energy.

Illustrative methods employing a surgical device comprising an expandable element (e.g., dilator 100a comprising the expandable element 203a) may include expanding and/or retracting the expandable element. FIGS. 17A, 18A, and 19A are isometric views of an exemplary membrane puncture procedure utilizing the dilator 100a, all according to at least some aspects of the present disclosure. For example, the expandable element 203a may be at least partially expanded after the initial opening 514 has been created, but before the expanded opening 516 has been created. Then, the dilator 100a may be advanced so that the expandable element 203a passes through the membrane 508, thereby creating a larger expanded opening 516. Alternatively, the dilator 100a may be advanced so that the expandable element 203a passes through the membrane 508 in the contracted configuration. Then, the expandable element 203a may be at least partially expanded and the dilator 100a may be pulled to withdraw the expandable element 203a through the expanded opening 516, thereby enlarging the expanded opening 516. Alternatively, the dilator 100a may be positioned such that the membrane 508 (e.g., the expanded opening 516) is at least partially in contact with the contracted expandable element 203a. Then, the expandable element 203a may be expanded to enlarge the expanded opening 516. Following use of the expandable element 203a, it may be contracted. A larger fenestration can be formed with the expandable element 203a. The delivery profile of the device can be minimized, while maximizing the dilation impact.

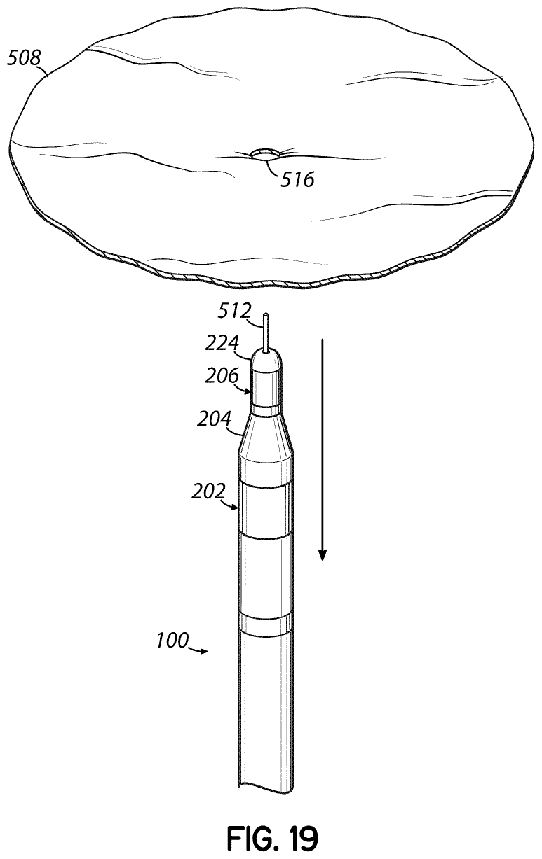

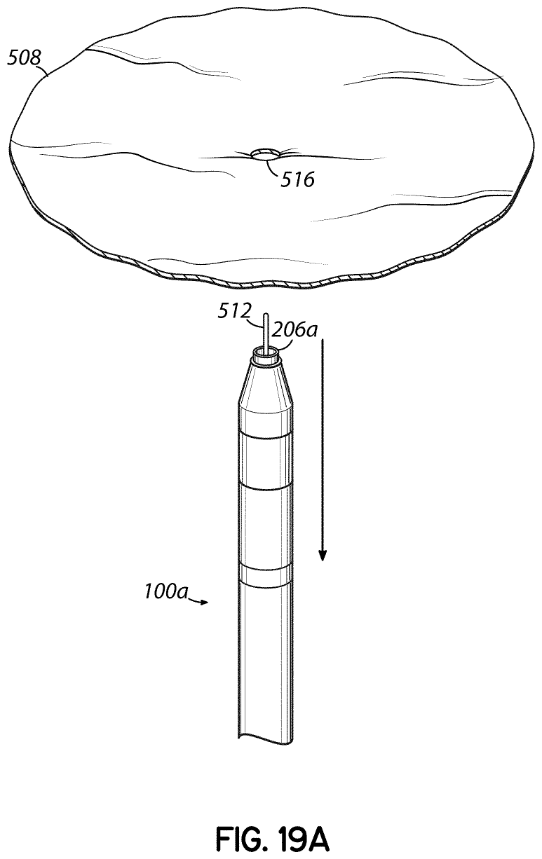

Referring to FIG. 19, the dilator 100 and/or the guidewire 512 may be withdrawn, leaving the expanded opening 516 through the lamina terminalis 508. The expanded opening 516 may allow drainage of CSF from the third ventricle 502, bypassing the stenosed aqueduct 504.

Illustrative methods of manufacturing a steerable dilator 100 may include assembling a steerable dilator 100 from a handle 300, a shaft 102, and an end effector 200. Illustrative methods may include constructing the handle 300, the shaft 102, and the end effector 200. Illustrative methods may include routing steering lines steering lines 212, 214, 218, 220 and/or power wires 122, 124 between the end effector 200 and the handle 300 through the shaft 102. Illustrative methods may include operatively connecting steering lines 212, 214, 218, 220 to steering actuators 304, 306 and/or steering rings 216, 222. Illustrative methods may include operatively connecting power wires 122, 124 to energizing elements 224, 226.

The present disclosure generally discloses “over-the-wire” techniques for performing a catheter based procedure. It will be understood that other catheter based techniques may be used instead for delivering instruments and components as generally described. These techniques may include, for example, “rapid exchange” techniques in which a catheter includes a short guide wire receiving sleeve or inner lumen extending through the distal portion of the catheter. The sleeve or inner lumen extends from a distal guide wire port in the distal end of the catheter to a proximal guide wire port spaced proximal to the proximal end of the dilation balloon. The proximal guide wire port is usually located between about 10 cm and about 50 cm from the distal guide wire port. A slit is provided in the catheter wall which extends from the second guide wire port, to a location proximal to the proximal end of the inflatable balloon to aid in the removal of the catheter from a guide wire upon withdrawal of the catheter from the patient. The structure of the rapid exchange catheter allows for the quick exchange of the catheter without the need for the use of an exchange wire or adding a guide wire extension to the proximal end of the guide wire. This type of catheter system may be used for similar benefits in procedures as generally shown and described in this disclosure.

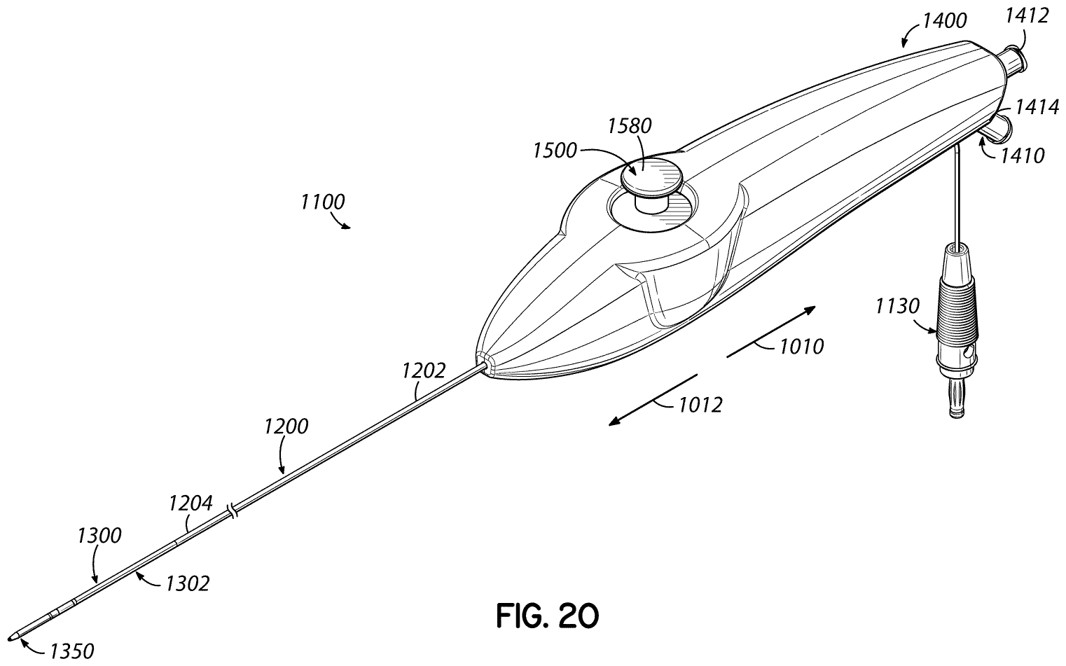

FIG. 20 is an isometric view of an illustrative steerable dilator 1100, according to at least some aspects of the present disclosure. The illustrative dilator 1100 includes an elongated shaft 1200 having a proximal end portion 1202 and a distal end portion 1204. As used herein to describe various embodiments from the perspective of a user of a surgical device, “proximal” may refer to a direction generally towards the user of the device, while “distal” may refer to a direction generally away from the user of the device. Similarly, in the context of a surgical device inserted into a patient's body and from the perspective of a user of the device, “proximal” may refer to a direction generally away from the center of the patient's body, and “distal” may refer to a direction generally towards the center of the patient's body. For reference, arrow 1010 points generally proximally and arrow 1012 points generally distally. The illustrative dilator 1100 includes an end effector 1300 disposed at the distal end portion 1204 of the shaft 1200, a handle 1400 disposed at the proximal end portion 1202 of the shaft 1200, a steering actuator 1500 disposed on the handle 1400, and a wiring harness 1130 configured to operatively connect a source of electrical energy (e.g., an electrosurgical generator) with the end effector 1300.

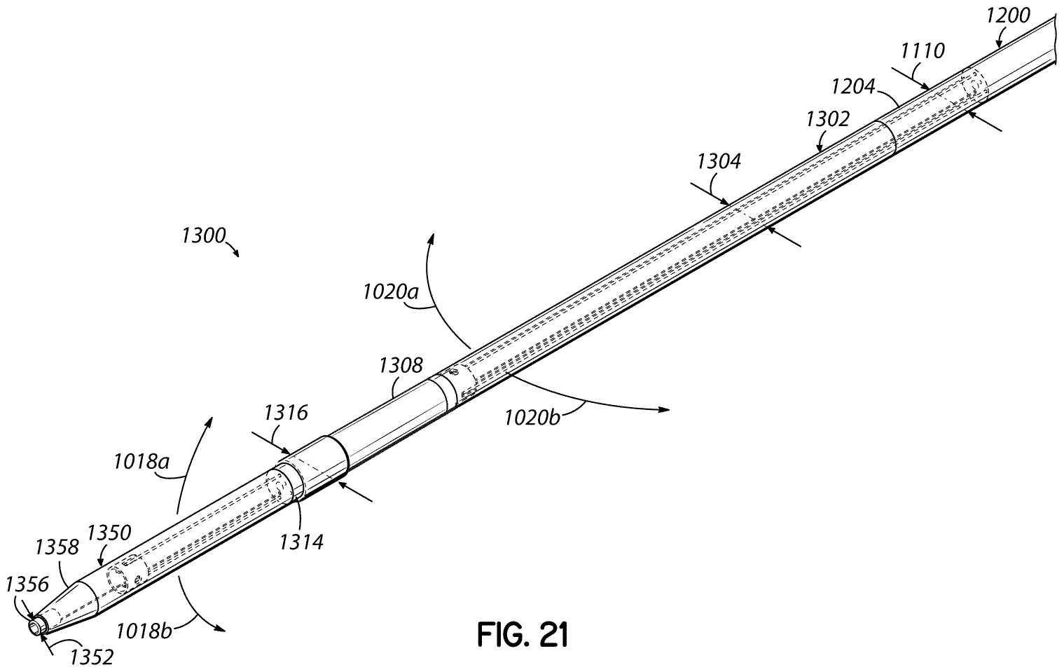

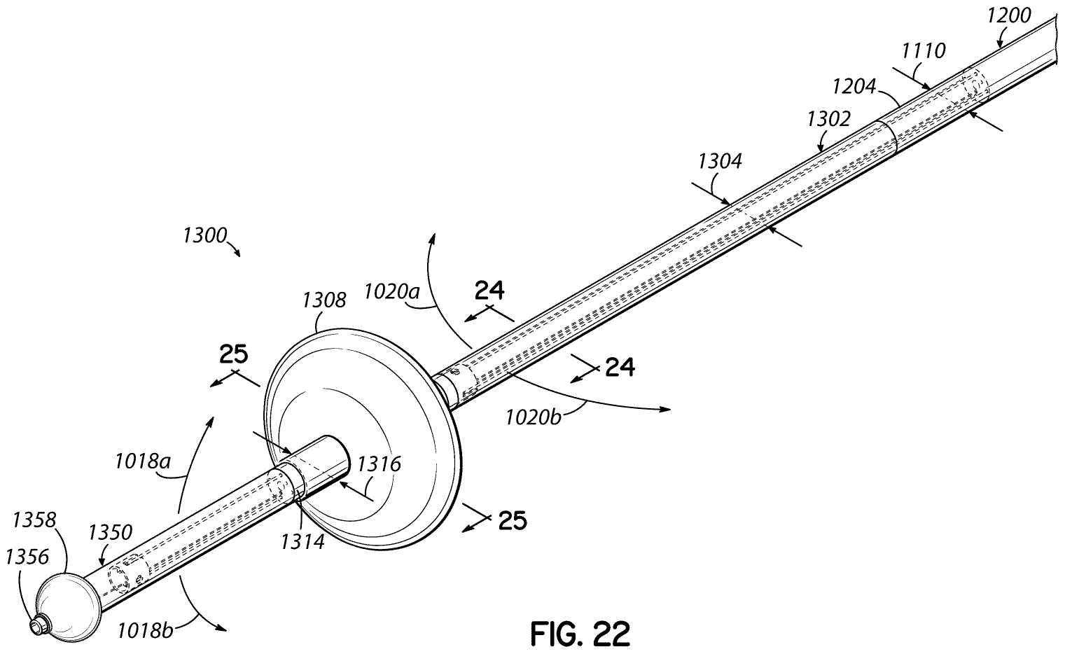

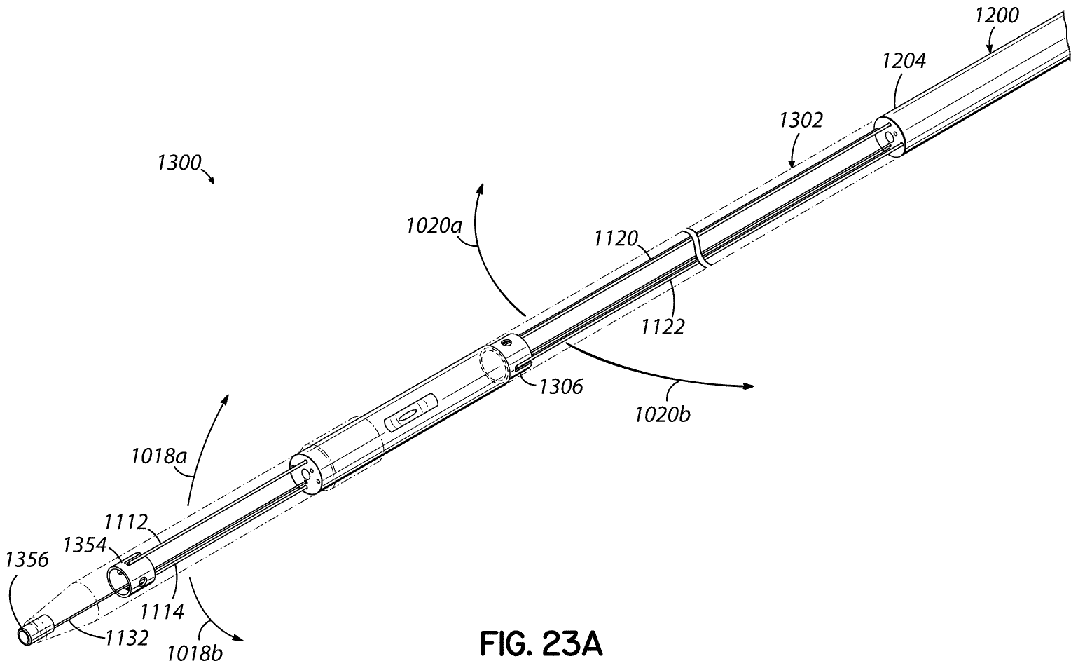

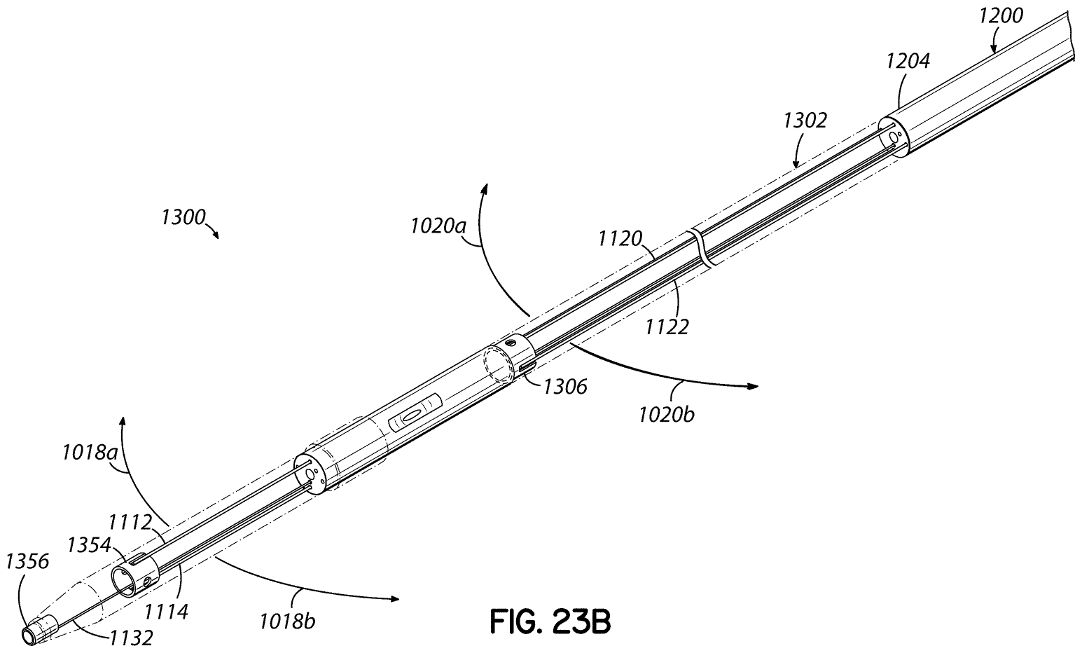

FIGS. 21 and 22 are isometric views of an illustrative end effector 1300 and FIGS. 23A and 23B are sectional views of an illustrative end effector 1300, according to at least some aspects of the present disclosure. The illustrative end effector 1300 includes a proximal dilation portion 1302 disposed proximate the distal end portion 1204 of the shaft 1200, and a distal tip portion 1350 disposed distally on the dilation portion 1302. The tip portion 1350 has a tip portion diameter 1352 and the dilation portion 1302 has a dilation portion diameter 1304. In some embodiments, the dilation portion diameter 1304 may approximately equal to the tip portion diameter 1352. In some embodiments, a shaft diameter 1110 may approximately equal the dilation portion diameter 1304.

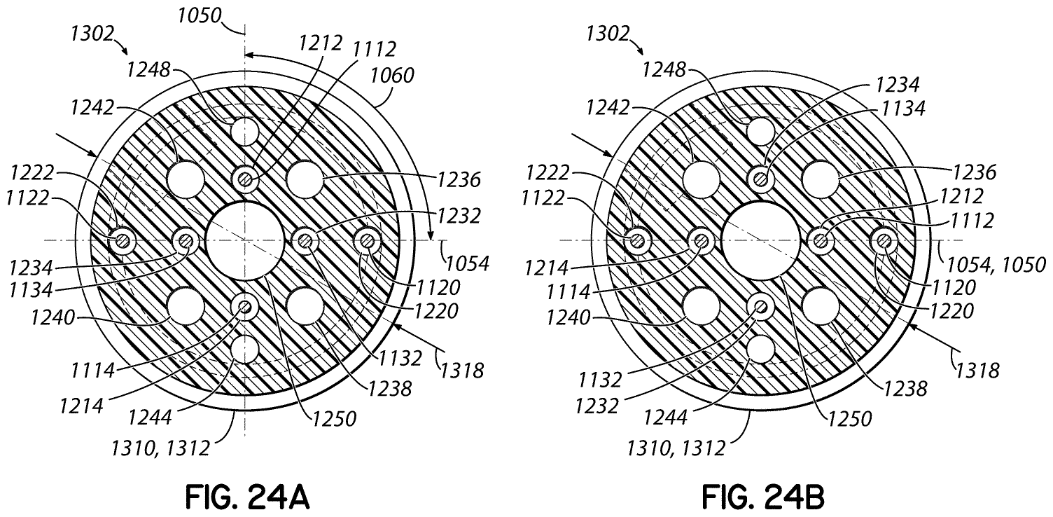

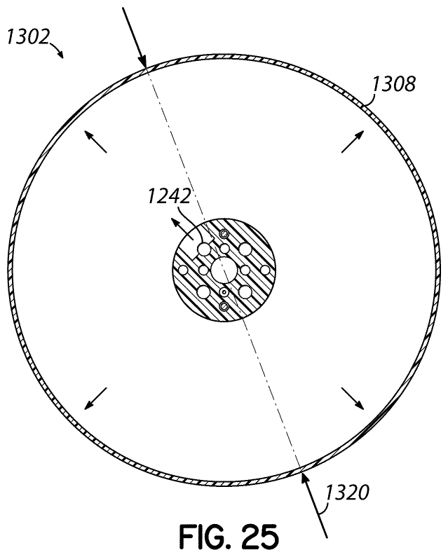

FIGS. 24A, 24B, and 25 are sectional views of an illustrative dilation portion 1302, according to at least some aspects of the present disclosure. Referring to FIGS. 22 through 25, the illustrative dilation portion 1302 includes an expandable element 1308, such as an inflatable element. The expandable element 1308 may be selectively expandable from approximately the expandable portion diameter 1316 to a fully expanded diameter 1320. In one exemplary embodiment, the expandable element 1308 may comprise a generally toroidal inflatable element configured to have an expanded diameter 1320. In other embodiments, the expandable element 1308 may comprise an inflatable element of a non-toroidal shape, for example. In some embodiments, the expandable element 1308 comprising an inflatable element may be generally in the form of a compliant balloon having a hardness of about Shore 60A to about Shore 25D. Expandable elements 1308, such as inflatable elements, may be fully inflated or partially expanded, such as by injecting and/or withdrawing fluid to achieve a desired partially or fully expanded diameter, for example. In some embodiments, the expandable element 1308 may be constructed of radiopaque materials, which may facilitate visualization of the dilator 1100 using various medical imaging modalities.

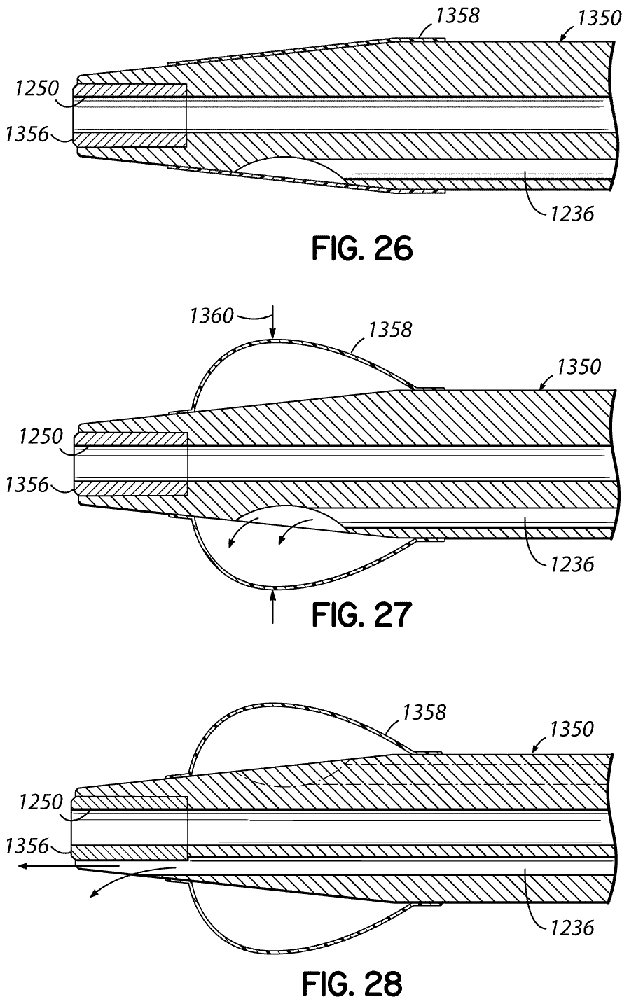

FIGS. 26 through 28 are sectional views of an illustrative tip portion 1350, according to at least some aspects of the present disclosure. Referring to FIGS. 22 through 23B and 26 through 28, the illustrative tip portion 1350 includes an expandable element 358, such as an inflatable element. The expandable element 1358 generally mimics the contour and diameter of the tip portion 1350. The expandable portion 1358 may be selectively expandable from approximately the tip portion diameter 1352 to a fully expanded diameter 1360. In one exemplary embodiment, the expandable element 1358 may comprise a generally teardrop shaped inflatable element configured to have an expanded diameter 1360. In other embodiments, the expandable element 1358 may comprise an inflatable element of a non-teardrop shape, for example. A balloon-like expansible element should be highly compliant to conform to and protect the anatomy during a procedure, for example. In some embodiments, the expandable element 1358 comprising an inflatable element may be generally in the form of a compliant balloon having a hardness of about Shore 60A to about Shore 25D. Expandable elements 1358, such as inflatable elements, may be fully inflated or partially expanded, such as by injecting and/or withdrawing fluid to achieve a desired partially or fully expanded diameter, for example. In some embodiments, the expandable element 1358 may be constructed of radiopaque materials, which may facilitate visualization of the dilator 1100 using various medical imaging modalities.

Referring to FIGS. 20 through 24B, the illustrative end effector 1300 is configured so that the tip portion 1350 is steerable in a first tip portion steering plane 1050 as shown by arrows 1018a and 1018b and the dilation portion 1302 is steerable in a first dilation portion steering plane 1054 as shown by arrows 1020a and 1020b. Generally, the tip portion 1350 is steerable relative to the dilation portion 1302 and the dilation portion 1302 is steerable relative to the shaft 1200. In some embodiments, only the tip portion 1350 may be steerable relative to the dilation portion 1302. In some embodiments, only the dilation portion 1302 may be steerable relative to the shaft 1200.

In some embodiments, the first dilation portion steering plane 1054 is disposed transversely relative to the first tip portion steering plane 1050. As used herein, “transverse” may refer to relative angular orientations that are non-parallel (e.g., perpendicular or oblique). For example, the first dilation portion steering plane 1054 may be disposed at a steering plane angle 1060 of about 90 degrees relative to the first tip portion steering plane 1050 as shown in FIGS. 21 and 24A. Such a configuration may be advantageous for some uses in which it may be desirable to utilize two-dimensional steering of the dilator 1100 relative to the longitudinal axis of the shaft 1200. In other embodiments, the steering plane angle 1060 may be greater than about 0 to about 90 degrees, for example. In some embodiments, the first tip portion steering plane 1050 and the first dilation portion steering plane 1054 may be substantially parallel, such as substantially coplanar (see FIGS. 23B and 24B).

Referring again to FIGS. 20 through 24B, the illustrative end effector 1300 is steerable through the action of one or more tension elements on one or more steering rings. The tip portion 1350 is steerable in the first tip portion steering plane 1050 by the selective proximal pulling of a first tip portion steering line 1112 and a second tip portion steering line 1114 connected to a tip portion steering ring 1354. Specifically, pulling proximally on the first tip portion steering line 1112 pulls on the tip portion steering ring 1354 so as to cause the tip portion 1350 to flex in the direction of arrow 1018a. Pulling proximally on the second tip portion steering line 1114 pulls on the tip portion steering ring 1354 so as to cause the tip portion 1350 to flex in the direction of arrow 1018b. The direction of arrow 1018a and the direction of arrow 1018b may be generally opposite directions. The tip portion steering lines 1112, 1114 may be connected to the tip portion steering ring 1354 by a weld or solder joint, or a mechanical fastener, for example. In some embodiments, the tip portion steering ring 354 may be constructed of a radiopaque material, which may facilitate visualization of the dilator 1100 using various medical imaging modalities.

Similarly, the dilation portion 1302 is steerable in the first dilation portion steering plane 1054 by the selective proximal pulling of a first dilation portion steering line 1120 and a second dilation portion steering line 1122 connected to a dilation portion steering ring 1306. Specifically, pulling proximally on the first dilation portion steering line 1120 pulls on the dilation portion steering ring 1306 so as to cause the dilation portion 1302 to flex in the direction of arrow 1020a. Pulling proximally on the second dilation portion steering line 1122 pulls on the dilation portion steering ring 1306 so as to cause the dilation portion 1302 to flex in the direction of arrow 1020b. The direction of arrow 1020a and the direction of arrow 1020b may be generally opposite directions. The dilation portion steering lines 1120, 1122 may be connected to the dilation portion steering ring 1306 by a weld or solder joint, or a mechanical fastener, for example. In some embodiments, the dilation portion steering ring 1306 may be constructed of a radiopaque material, which may facilitate visualization of the dilator 1100 using various medical imaging modalities.

As described below, the tension elements (e.g., steering lines 1112, 1114, 1120, 1122) are tensioned by one or more actuators disposed on the handle 1400. Generally, the tension elements (e.g., steering lines 1112, 1114, 1120, 1122) may be constructed of any materials capable of transmitting a tensile force from the actuators to the steering rings 1306, 1350. In some embodiments, the steering lines 1112, 1114, 1120, 1122 may comprise metal wires and/or various suture materials.

Referring to FIGS. 20 through 24B, the shaft 1200 and end effector 1300 may include one or more longitudinal lumens extending from the proximal end portion 1202 of the shaft 1200 to the distal end portion 1204 of the shaft 1200 and through the end effector 1300 to the tip portion 1350. In the illustrative embodiment, each of the steering lines 1112, 1114, 1120, 1122 is slidably disposed within a respective steering line lumen 1212, 1214, 1220, 1222 in the shaft 1200 wherein the steering lines 1112, 1114, 1120, 1122 run from the steering actuator 1500 through the shaft 1200 to the respective steering rings 1354, 1306. In this exemplary embodiment, the lumens of each respective pair of steering line lumens 1212, 1214, and 1220, 1222 are positioned substantially diametrically opposite each other (e.g., about 180 degrees relative to each other). Alternative embodiments may comprise only a single steering line 1112, 1114, 1120, 1122 and/or steering line lumen 1212, 1214, 1220, 1222 for a particular steerable portion 1350, 1302, which may provide unidirectional steering for that steerable portion 1350, 1302.

Referring to FIGS. 20 through 22, 24A, 24B, and 25 through 28, in this illustrative embodiment, the expandable element 1308 is configured to be selectively expanded and/or contracted, such as by injecting or withdrawing fluid (e.g., a liquid or a gas) via an inflation connection 1414. The inflation connection 1414 is provided on a connection hub 1410 at the proximal end of the handle 1400, such as on a dedicated branch of the connection hub 1410. The expandable element 1358 is configured to be selectively expanded and/or contracted, such as by injecting or withdrawing fluid (e.g., a liquid or a gas) via an inflation connection 1414. The inflation connection 1414 is provided on a connection hub 1410 at the proximal end of the handle 1400, such as on a dedicated branch of the connection hub 1410. The dedicated branch of the connection hub 1410 may be operably connected to the expandable element 1308 through a lumen 1242 in the shaft 1200 and end effector 1300, for example. The dedicated branch of the connection hub 1410 may be operably connected to the expandable element 1358 through a lumen 1236 in the shaft 1200 and end effector 1300, for example.

Referring to FIGS. 20, 21, 23A, 24A, and 24B the illustrative end effector 1300 includes one or more energizing elements, such as a tip energizing element 1356, which forms the distal end of the tip portion 1350. The energizing element 1356 of the distal tip portion 1350 is configured to receive electrical energy via the wiring harness 1130 and is configured to deliver the electrical energy to biological tissues. The illustrative wiring harness 1130 may include one or more tip portion power wires 1132, 1134 extending through lumens 1232, 1234 to the tip energizing element 1356. Depending on the electrosurgical technique that is employed, the power wires 1132, 1134 may comprise one or more separate and/or insulated conductors. The power wires 1132, 1234 may be connected to the energizing element 1356 by solder connections, for example. In some embodiments, the energizing element 1356 may be constructed of radiopaque materials, which may facilitate visualization of the dilator 1100 using various medical imaging modalities.

In the illustrative embodiment, the distal end of the tip portion 1350 is defined at least partially by the tip energizing element 1356. The tip energizing element 1356 is shaped to facilitate passage of the tip portion 1350 through a biological tissue membrane. The tip energizing element 1356 may include a beveled shape (e.g., generally beveled) as shown and/or other shapes, such as radiuses and/or tapers (e.g., generally rounded or pointed).

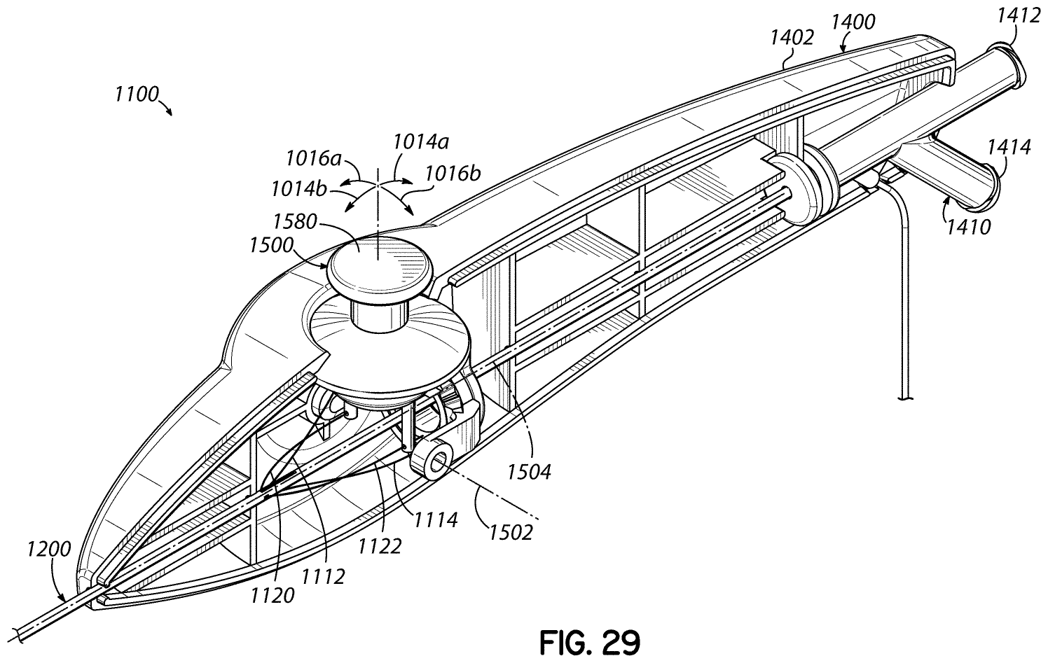

FIG. 29 is an isometric sectional view of an illustrative steerable dilator handle 1400, according to at least some aspects of the present disclosure. The handle 1400 comprises a handle body 1402 generally configured to be gripped by a user's hand. The handle body 1402 may be constructed of a generally rigid polymer (e.g., Shore 75D or higher). The illustrative steering actuator 1500 is rotatably disposed on the handle 1400. The steering actuator 1500 may be moved by the user in relation to the handle 1400 in any or all directions indicated by arrows 1014a, 1014b, 1016a, and 1016b. The shaft 1200 is disposed on the handle 1400 and passes through a portion of the steering actuator 1500.

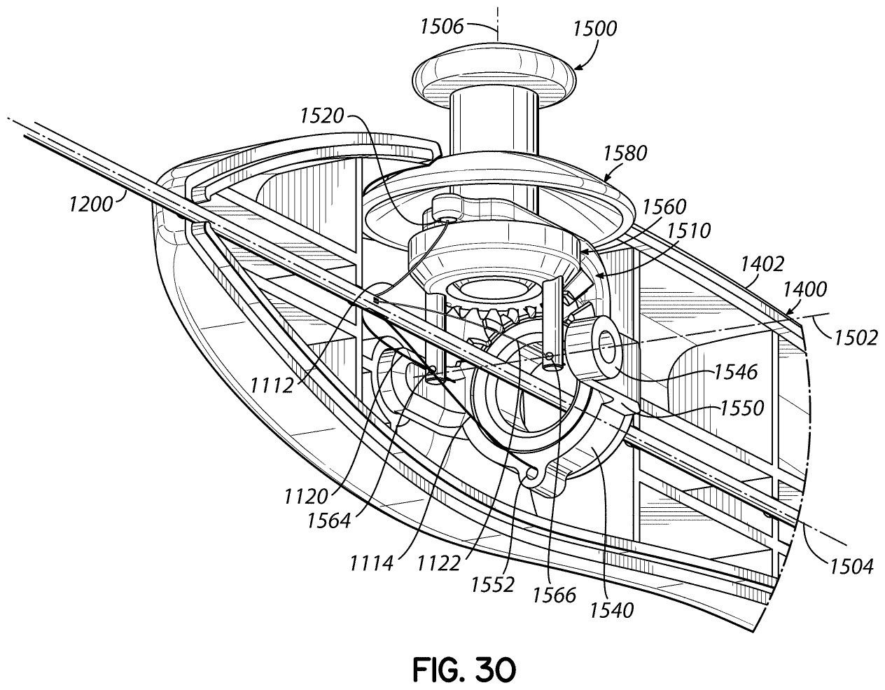

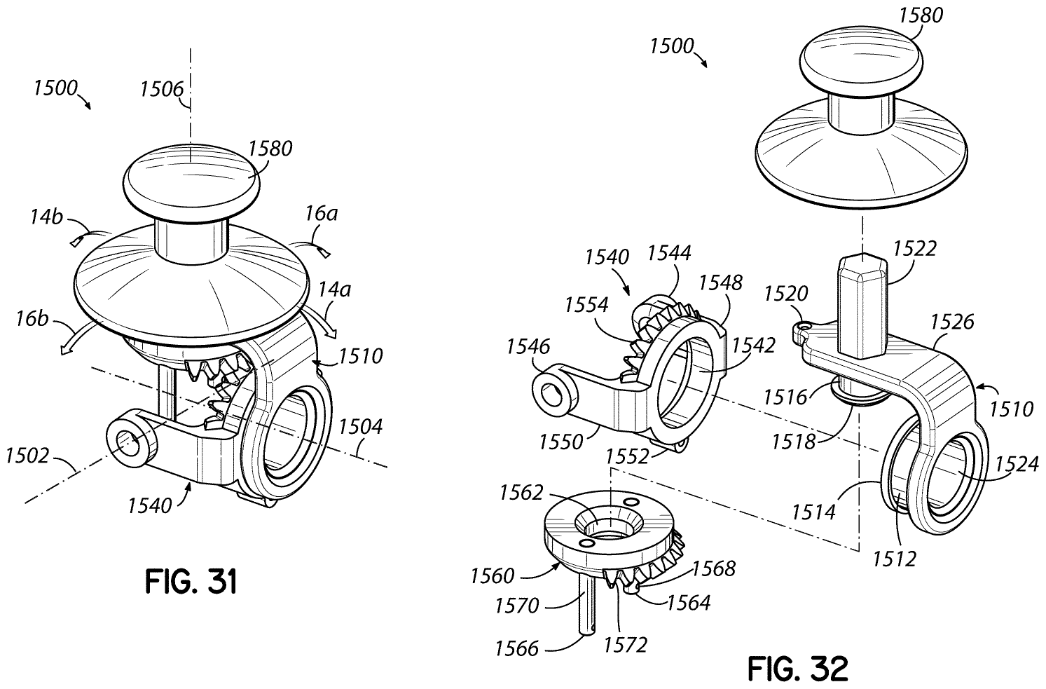

FIG. 30 is a detailed isometric cutaway view of an illustrative handle 1400, FIG. 31 is a detailed isometric view of an illustrative steering actuator 1500, and FIG. 32 is an exploded view of the illustrative steering actuator 1500, all according to at least some aspects of the present disclosure.

Referring to FIGS. 20 and 29 through 32, the illustrative handle 1400 includes a steering actuator 1500. In this exemplary embodiment, the steering actuator 1500 is generally a pair of gimbals or pivoted supports that allow for the rotation of the steering actuator 1500 about a first axis of rotation 1502 and a second axis of rotation 1504 relative to the handle 1400. In this illustrative example the second axis of rotation 1504 is generally along the length of the shaft 1200 and the first axis of rotation 1502 is generally perpendicular to the second axis of rotation 1504. The steering actuator 1500 is rotatably disposed on the handle 1400 for operation by the user.

In this exemplary embodiment, the steering actuator 1500 includes an actuator body 1510, a yoke 1540, a rotational actuator 1560, and an actuator handle 1580. The actuator body 1510 includes a yoke shaft portion 1512 and yoke retainer portion 1514. The yoke 1540 includes a round hole 1542. The actuator body 1510 is rotatably mounted to the yoke 1540 with the yoke shaft portion 1512 passing through the hole 1542 and the actuator body 1510 is secured to the yoke 1540 by means of the yoke retainer portion 1514. The yoke 1540 includes a pair of pivot shafts 1544, 1546. The pivot shafts 1544, 1546 of the yoke 1540 are rotatably mounted on the handle 1400. The actuator body 1510 includes a rotational actuator shaft portion 1516 and a rotational actuator retainer portion 1518 for rotatably mounting the rotational actuator 1560. The rotational actuator 1560 includes a round hole 1562. The rotational actuator 1560 is rotatably mounted to the actuator body 1510 with the rotational actuator shaft portion 1516 passing through the hole 1562 and the rotational actuator 1560 is secured to the actuator body 1510 by means of the rotational actuator retainer portion 1518. The actuator body 1510 includes an actuator handle shaft portion 1522 for mounting the actuator handle 1580. The yoke 1540 includes a tooth portion 1554 arranged to engage an opposed tooth portion 1572 on the rotational actuator 1560. In this illustrative embodiment, the tooth portions 1554, 1572 act as a matched pair of beveled gears.

In this illustrative embodiment, the steering actuator 1500 is disposed on the handle 1400 such that the actuator handle 1580 may be moved by the user about the two axis of rotation 1502, 1504 by moving the actuator handle 1580 in the directions defined by the arrows 1014a, 1014b and 1016a, 1016b respectively. Moving the actuator handle 1580 in the direction of arrows 1014a and 1014b rotates the steering actuator 1500 about the axis of rotation 1502 with the pivot shafts 1544, 1546 of the yoke 1540 rotating relative to the handle 1400. Moving the actuator handle 1580 in the direction of arrows 1016a and 1016b rotates the actuator body 1510 about the axis of rotation 1502 with the yoke shaft portion 1512 rotating in the yoke 1540. Further, moving the actuator handle 1580 in the direction of arrows 1016a and 1016b causes the tooth portion 1554 on the yoke 1540 to apply a force to the tooth portion 1572 on the rotational actuator 1560 causing the rotational actuator 1560 to rotate on the rotational actuator shaft portion 1518 of the actuator body 1510 about an axis of rotation 1506.

In this illustrative embodiment, the steering actuator 1500 includes four steering line attachment points 1520, 1552, 1564, 1566. The actuator body 1510 includes steering line attachment point 1520. The yoke 1540 includes steering line attachment point 1552. The rotating actuator includes steering line shafts 1568, 1570 which include steering line attachment points 1564, 1566 respectively.

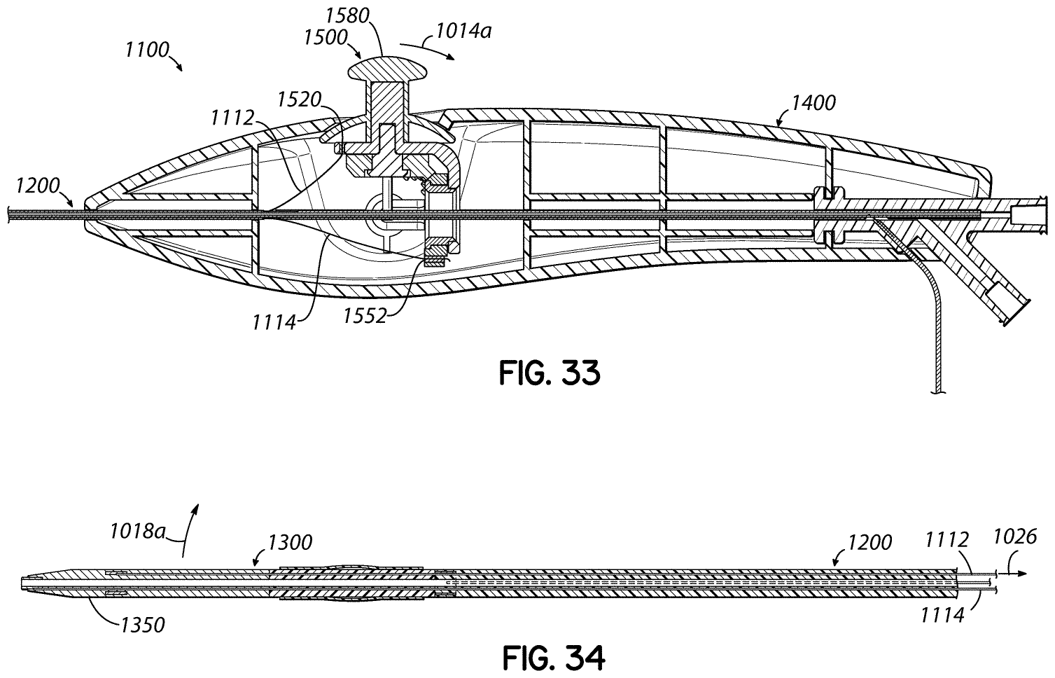

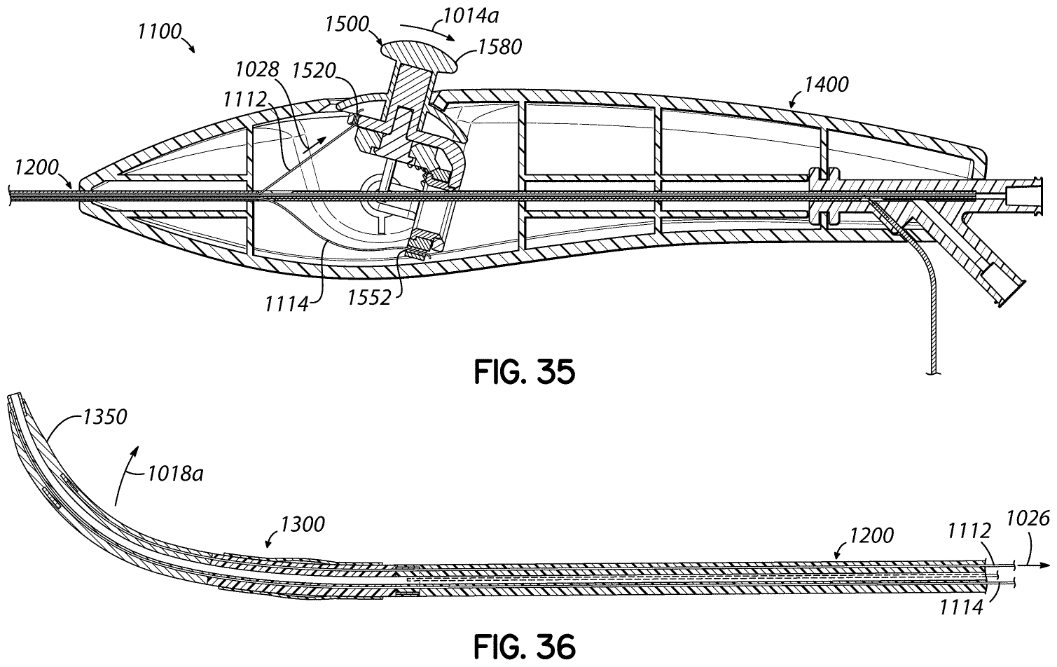

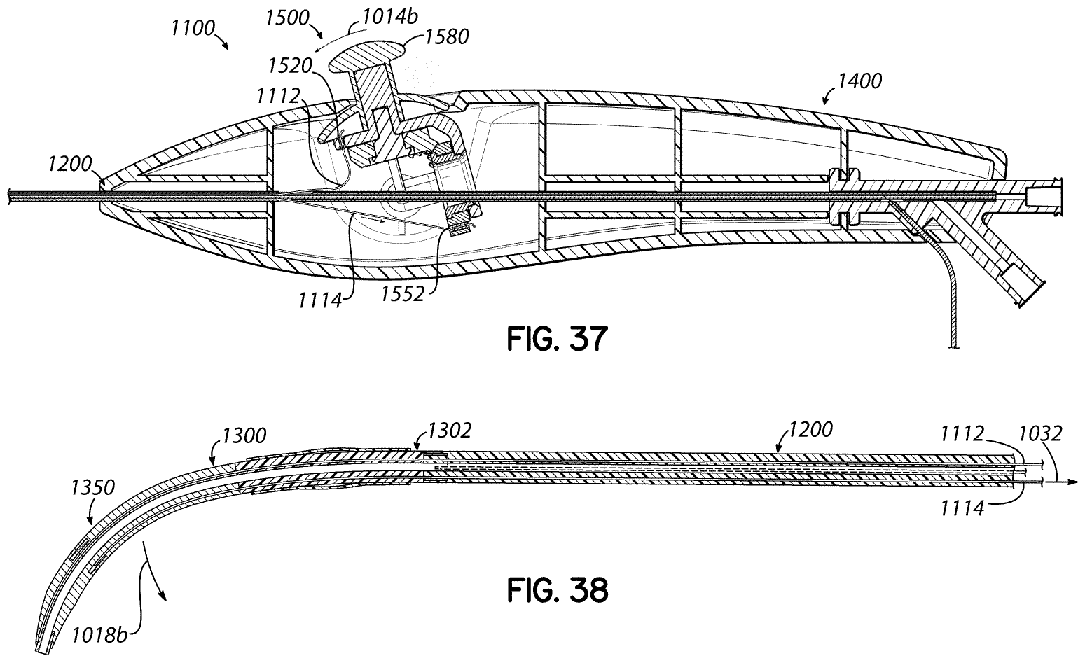

FIGS. 33, 35, and 37 are sectional views of an illustrative handle 1400, shaft 1200, and steering actuator 1500, and FIGS. 34, 36, and 38 are sectional views of an illustrative end effector 1300 and shaft 1200, according to at least some aspects of the present disclosure.

Referring to FIGS. 20, 21, 23A through 24B, 29, 30, and 33 through 38, the illustrative tip portion 1350 of the end effector 1300 is steerable through the action of one or more tension elements 1112, 1114 and the illustrative steering actuator 1500. In this illustrative embodiment the first tip portion steering line 1112 is connected to the steering line attachment point 1520 and the second tip portion steering line 1114 is connected to the steering line attachment point 1552. The tip portion steering lines 1112, 1114, may be connected to the attachment points 1520, 1552 by a weld or solder joint, or a mechanical fastener, for example.

In this illustrative embodiment, the actuator handle 1580 may be moved by the user about the axis of rotation 1502 by moving the actuator handle 1580 in the directions defined by the arrows 1014a and 1014b, see FIGS. 29, 33, 35, and 37. Moving the actuator handle 1580 in the direction of arrows 1014a and 1014b rotates the steering actuator 1500 about the axis of rotation 1502 thereby alternatively creating tension on the tip portion steering lines 1112, 1114. By moving the actuator handle 1580 in the direction defined by the arrow 1014a the steering actuator 1500 creates tension on the tip portion steering line 1112 in the direction of arrows 1026 and 1028 thereby moving the tip portion 1350 in the first tip portion steering plane 1050 in the direction defined by arrow 1018a. By moving the actuator handle 1580 in the direction defined by the arrow 1014b the steering actuator 1500 creates tension on the tip portion steering line 1114 in the direction of arrows 1030 and 1032 thereby moving the tip portion 1350 in the first tip portion steering plane 1050 in the direction defined by arrow 1018b.

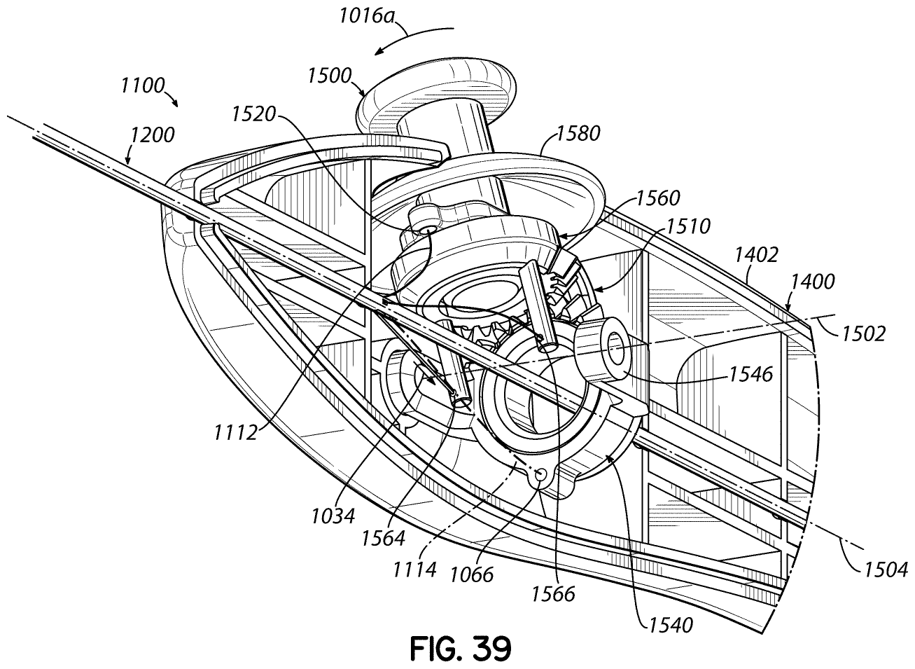

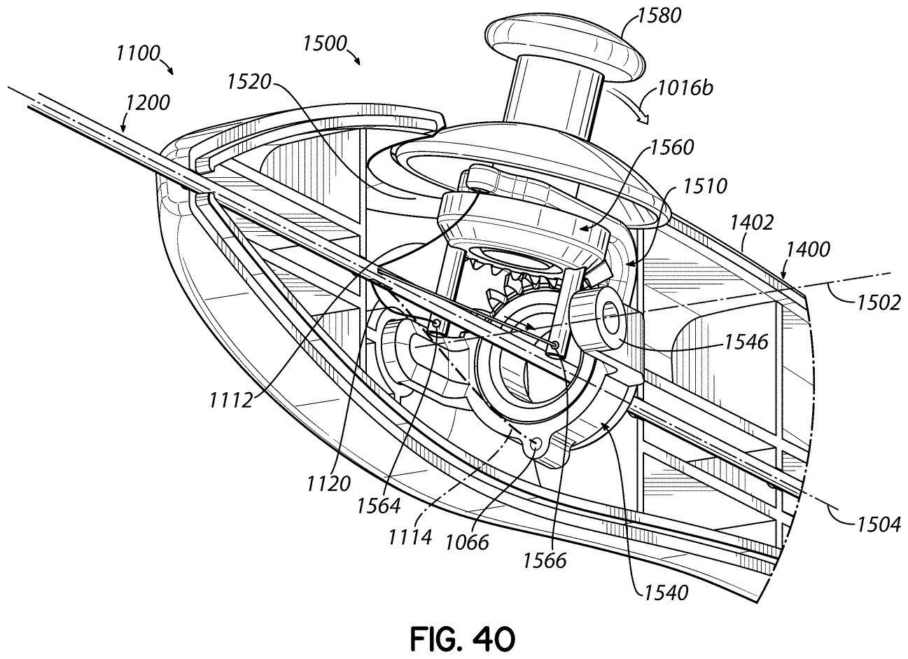

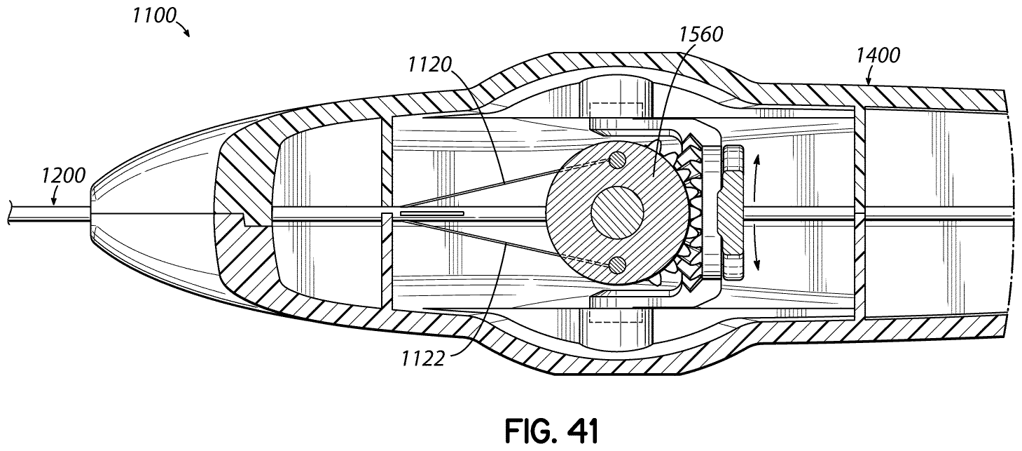

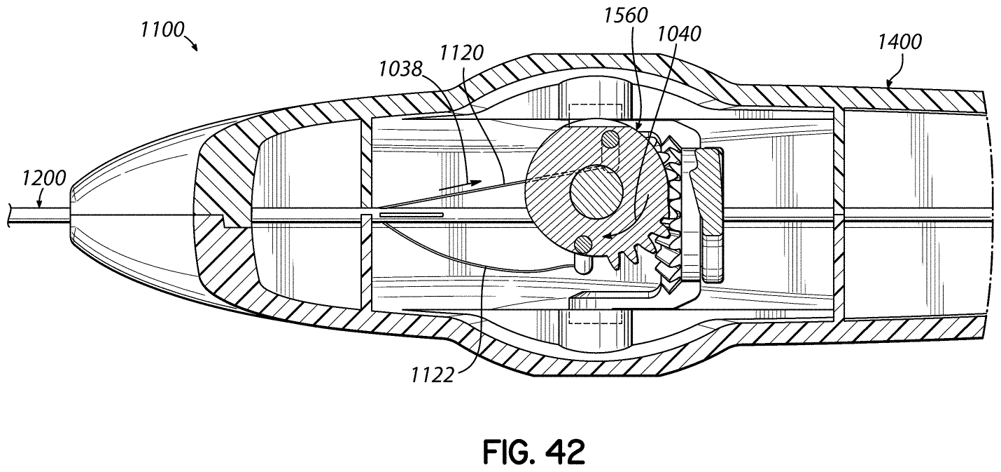

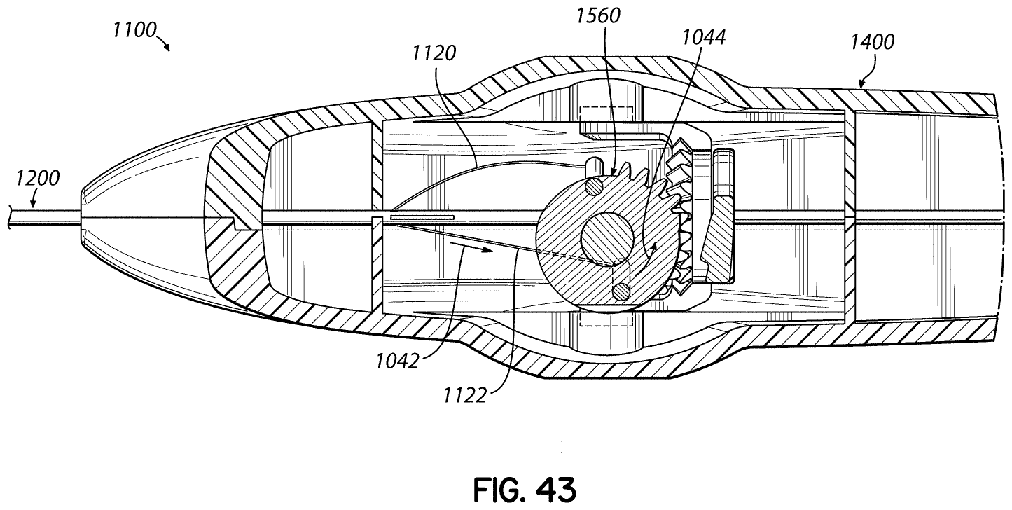

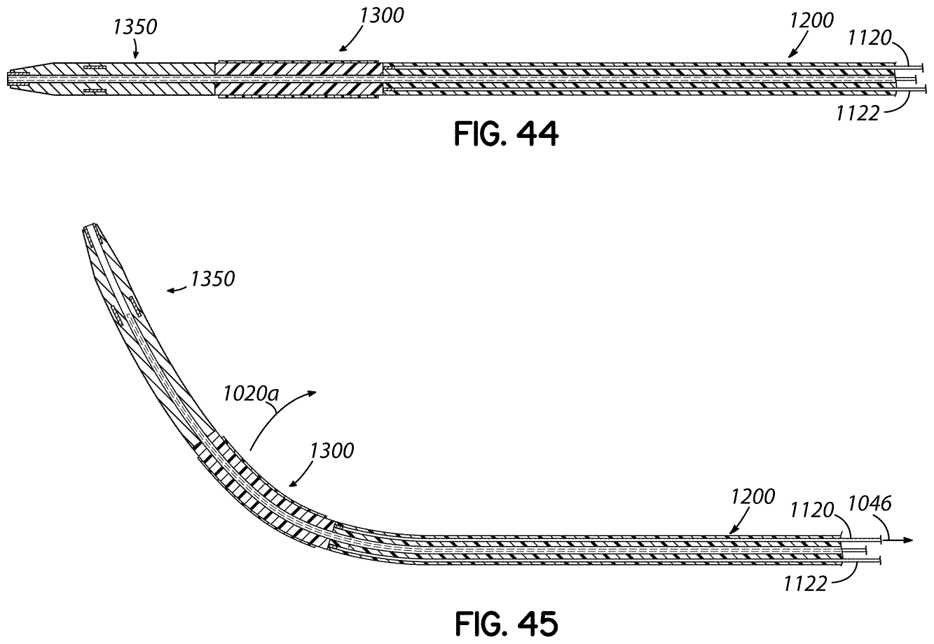

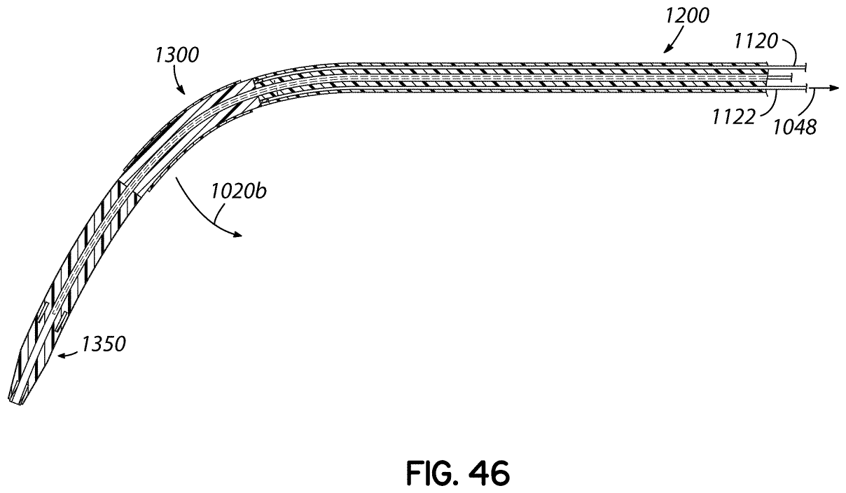

FIGS. 39 and 40 are isometric sectional views of an illustrative handle 1400, shaft 1200, and steering actuator 1500, FIGS. 41, 42, and 43 are sectional views of an illustrative handle 1400, shaft 1200, and steering actuator 1500, and FIGS. 44, 45, and 46 are sectional views of an illustrative end effector 1300 and shaft 1200, according to at least some aspects of the present disclosure.

Referring to FIGS. 20, 21, 23A through 24B, 29, 30, and 39 through 46, the illustrative dilation portion 1302 of the end effector 1300 is steerable through the action of one or more tension elements and the illustrative steering actuator 1500. In this illustrative embodiment the first dilation portion steering line 1120 is connected to the steering line attachment point 1564 and the second dilation portion steering line 1122 is connected to the steering line attachment point 1566. The dilation portion steering lines 1120, 1122, may be connected to the attachment points 1564, 1566 by a weld or solder joint, or a mechanical fastener, for example.

In this illustrative embodiment, the actuator handle 1580 may be moved by the user about the axis of rotation 1504 by moving the actuator handle 1580 in the directions defined by the arrows 1016a and 1016b, see FIGS. 29, 39, and 40. Moving the actuator handle 1580 in the direction of arrows 1016a and 1016b rotates the steering actuator 1500 about the axis of rotation 1504 thereby alternatively creating tension on the dilation portion steering lines 1220, 1222. By moving the actuator handle 1580 in the direction defined by the arrow 1016a the steering actuator 1500 creates tension on the dilation portion steering line 1220 in the direction of arrows 1034, 1046 thereby moving the dilation portion 1302 in the first dilation portion steering plane 1054 in the direction defined by arrow 1020a. By moving the actuator handle 1580 in the direction defined by the arrow 1016b the steering actuator 1500 creates tension on the dilation portion steering line 1122 in the direction of arrows 1036, 1048 thereby moving the dilation portion 1302 in the first dilation portion steering plane 1054 in the direction defined by arrow 1020b. A user may be able grasp the handle 1400 with one hand and operate the actuator handle 1580 with a thumb of said hand allowing for a single hand operation of the steering actuator 1500, for example

Referring to FIGS. 20, 21, 23A, 23B, 24A, and 24B the proximal end portion of the handle 1400 includes a proximally extending portion of the wiring harness 1130, including power wires 1132, 1134. Additionally, in this illustrative embodiment, the proximal end portion of the handle 1400 includes the proximal end of the guidewire lumen 1250. One or more mechanical couplings 1412, 1414, such as female Luer-lock fittings are provided on the guidewire lumen 1250 to facilitate, for example, attachment of a syringe for injection of fluid (e.g., saline, contrast) through the guidewire lumen 1250. The couplings 1412, 1414, may be used in connection with insertion of a guidewire and/or to attach a sealing device to prevent fluid leakage via the guidewire lumen 1250.

The proximally extending portion of the wiring harness 1130 is positioned in the handle 1400 to avoid conflicting with the guidewire lumen 1250 and the one or more mechanical couplings 1412, 1414. In some embodiments, the wires 1132, 1134 of the wiring harness 1130 may be color-coded, which may reduce the risk of user confusion. In this illustrative embodiment, a guidewire lumen 1250 extends longitudinally through the end effector 1300, shaft 1200, and handle 1400, and is configured to slidably receive a guidewire therethrough. In the illustrative embodiment, the guidewire lumen 1250 is substantially centrally located in the shaft 1200 and the end effector 1300.

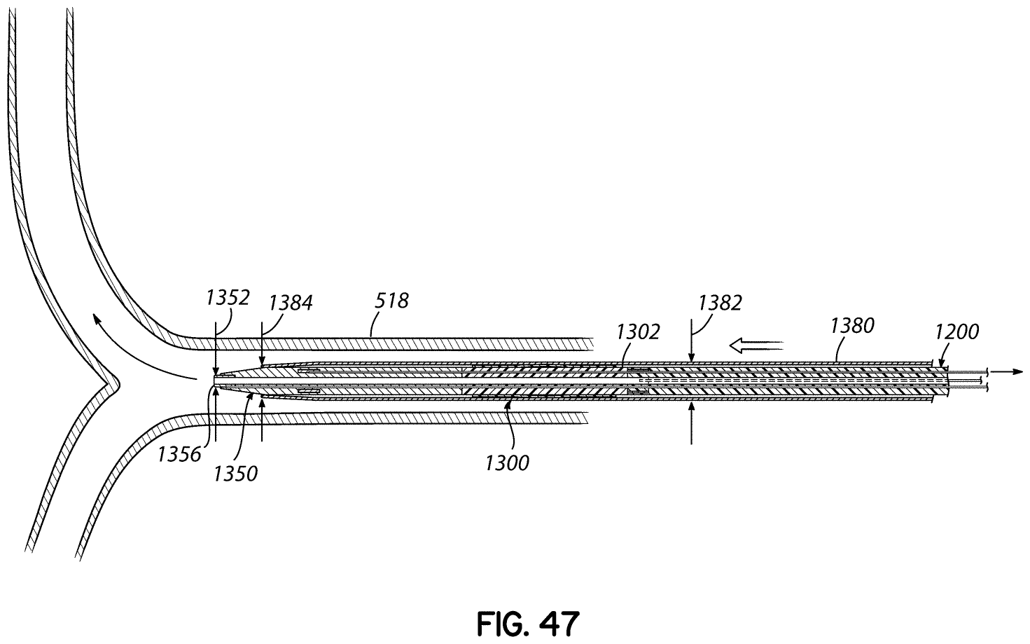

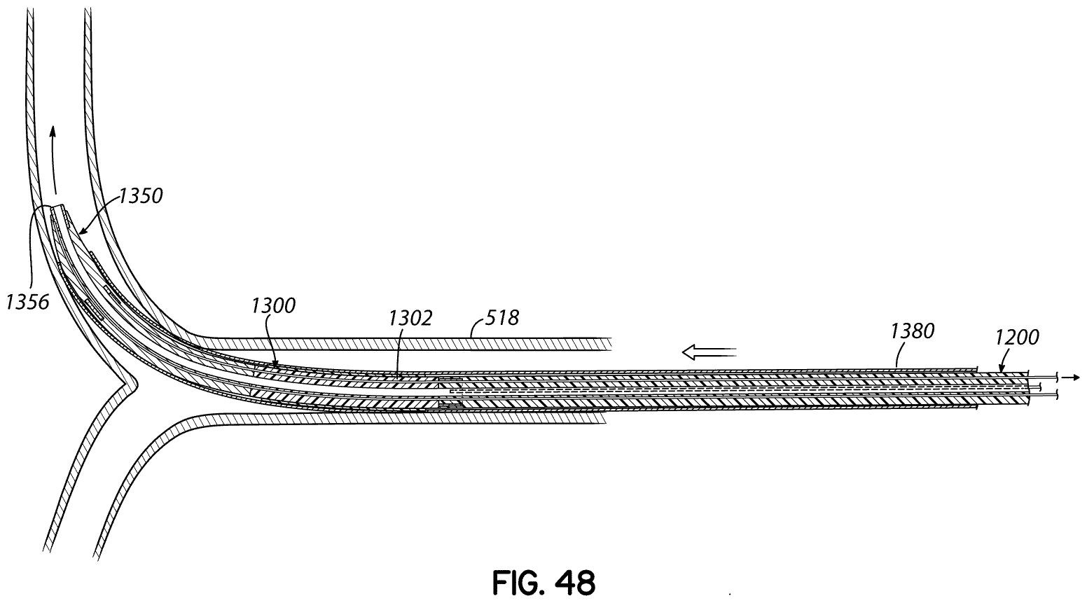

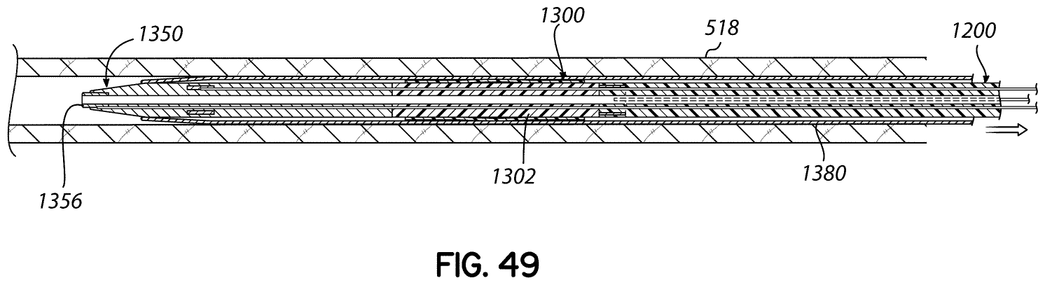

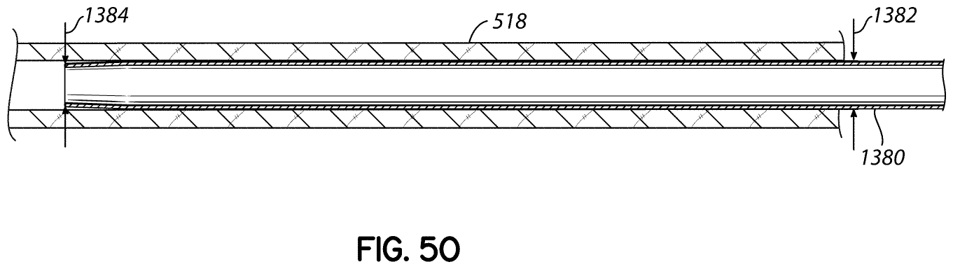

FIGS. 47, 48, and 49, are sectional views of an illustrative stent 1380 disposed on the end effector 1300 inside a vessel 518. FIG. 50 is a sectional view of an illustrative stent 1380 in position inside a vessel 518 after a procedure, for example. In this illustrative example the stent 1380 is generally cylindrical and generally mimics the contour and diameter of the end effector 1300. The stent 1380 is configured to fit over the end effector 1300. The stent 1380 is generally tapered at the distal end. The stent 1380, has a stent diameter 1382, a distal end diameter 1384. The stent diameter 1382 is greater than the tip portion diameter 1352 and the distal end diameter 1384 is less than the expandable portion diameter 1316 (see FIG. 21). In this illustrative example the stent 1380 is generally flexible for steerability with the end effector 1300. In this illustrative example, with the stent 1380 on the end effector 1300 the stent 1380 may be directed to a location, such as inside a vessel 518 for example, by the user utilizing the dilator 1100. Once in a location determined by the user, the dilator 1100 may be withdrawn from the stent 1380 leaving the stent 1380 in said location.

Illustrative methods of employing a surgical device comprising an stent 1380 disposed on the end effector 1300 may include guiding the end effector 1300 to a location by the user utilizing the dilator 1100. Once in a location determined by the user, the dilator 1100 may be withdrawn from the stent 1380 leaving the stent 1380 in said location.

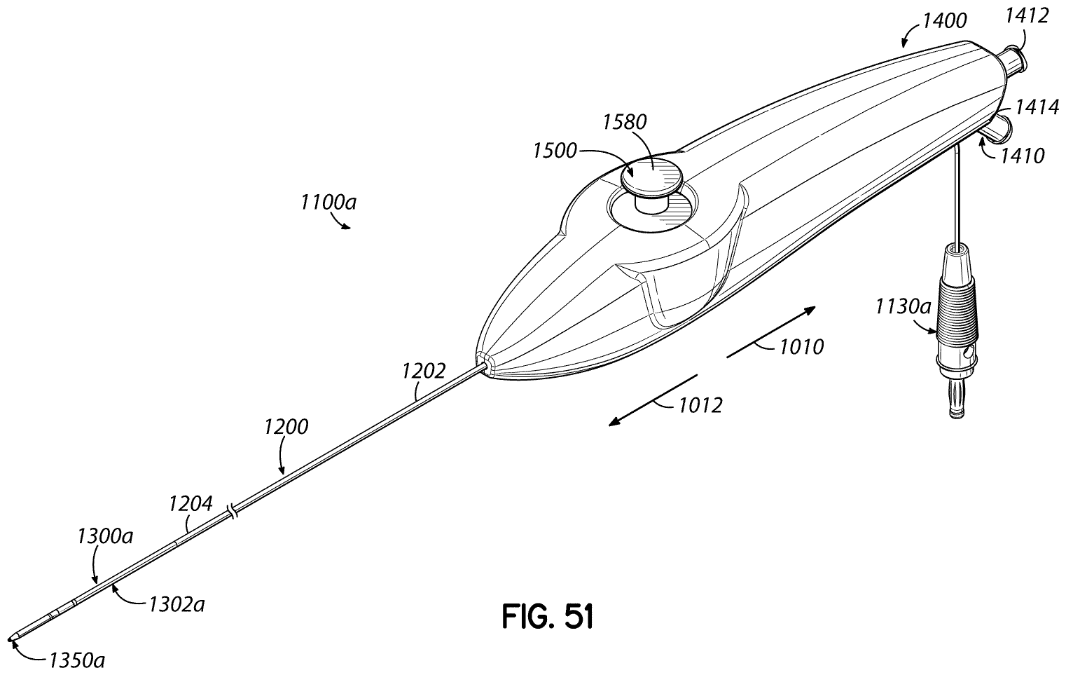

FIG. 51 is an isometric view of an alternate illustrative steerable dilator 1100a, according to at least some aspects of the present disclosure. Generally, the steerable dilator 1100a is similar in construction and operation to the steerable dilator 1100 described above and any feature(s) of the steerable dilator 1100a may be used in various other exemplary embodiments according to the present disclosure. Like reference numbers refer to like components and attendant function. For brevity, the following description omits redundant description and focuses on the differences between the steerable dilator 1100a and the steerable dilator 1100. The illustrative dilator 1100a includes an optical fiber harness 1130a configured to operatively connect a source of light energy with the end effector 1300a.

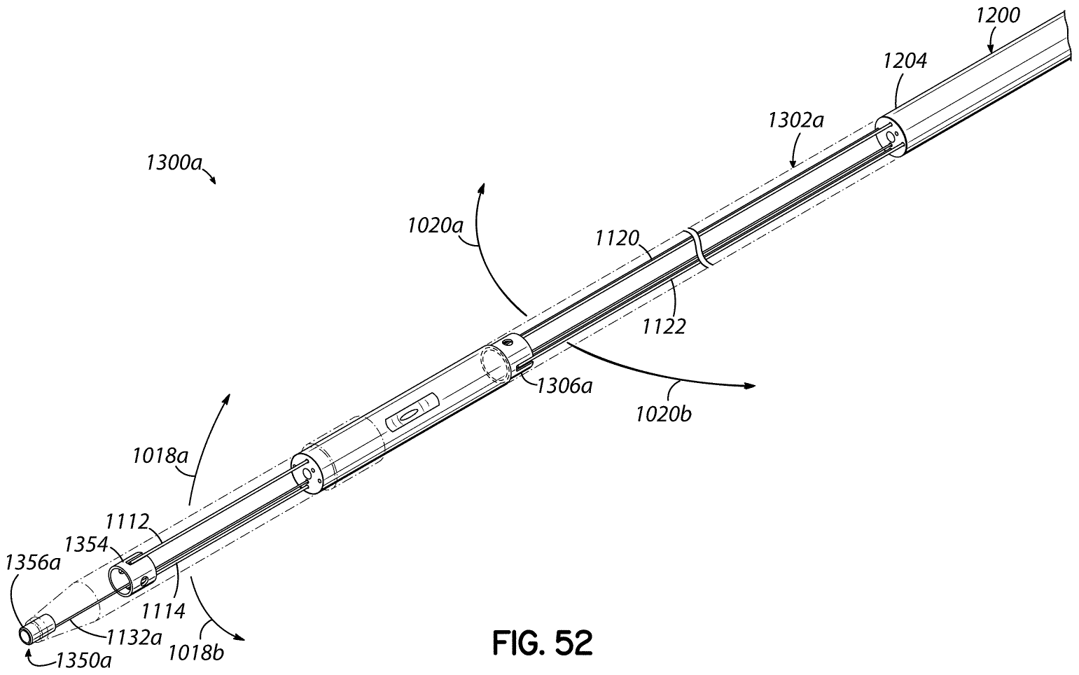

FIG. 52 is a sectional view of an alternate illustrative end effector 1300a similar to the end effector 1300 described above and according to at least some aspects of the present disclosure. The illustrative end effector 1300a includes a distal tip portion 1350a disposed distally on the dilation portion 1302a.

Referring to FIGS. 51, and 52, the illustrative end effector 1300a includes one or more light elements, such as light element 1356a, which forms the distal end of the tip portion 1350a. As described below, the light element 1356a of the distal tip portion 1350a is configured to receive light via an optical fiber harness 1130a and is configured to deliver the light to biological tissues. The illustrative optical fiber harness 1130a includes a tip portion optical fiber element 1132a extending through the shaft 1200 to the light element 1356a. Depending on the surgical technique that is employed, the optical fiber element 1132a may comprise one or more separate optical fibers. In some embodiments, the light element 1356a may be constructed of radiopaque materials, which may facilitate visualization of the dilator 1100a using various medical imaging modalities. The light element 1356a may be used on other catheters as well as the dilator described above.

In the illustrative embodiment, the distal end of the tip portion 1350a is defined at least partially by the light element 1356a. The light element 1356a is shaped to facilitate passage of the tip portion 1350a through a biological tissue membrane. The tip portion 1350a may include a beveled shape (e.g., generally beveled) as shown and/or other shapes, such as radiuses and/or tapers (e.g., generally rounded or pointed).

Illustrative methods employing a surgical device comprising a light element 1356a, which forms the distal end of the tip portion 1350a may include guiding the tip portion 1350a to a location by the user utilizing the dilator 1100a. Once in a location determined by the user, the light element 1356a may be energized by the user to apply light therapy to biological tissues such as for instance brain or spinal tissue.

Referring to FIGS. 1-10, an illustrative method of manufacturing a steerable dilator 100 may include assembling a steerable dilator 100 from a handle 300, a shaft 102, and an end effector 200. A method of manufacture may further include constructing the handle 300, the shaft 102, and the end effector 200. Methods of manufacture may include routing steering lines 212, 214, 218, 220 and/or power wires 122, 124 between the end effector 200 and the handle 300 through the shaft 102. A method of manufacture may include operatively connecting steering lines 212, 214, 218, 220 to the steering actuators 304, 306 and/or steering rings 216, 222. Methods of manufacture may include operatively connecting power wires 122, 124 to the energizing elements 224, 226.

While the present invention has been illustrated by the description of specific embodiments thereof, and while the embodiments have been described in considerable detail, it is not intended to restrict or in any way limit the scope of the appended claims to such detail. The various features discussed herein may be used alone or in any combination within and between the various embodiments. Additional advantages and modifications will readily appear to those skilled in the art. The invention in its broader aspects is therefore not limited to the specific details, representative apparatus and methods and illustrative examples shown and described. Accordingly, departures may be made from such details without departing from the scope or spirit of the general inventive concept.

What is claimed is:

1. A steerable dilator comprising:
an elongated shaft including a proximal end portion and a distal end portion;
an end effector disposed at the distal end portion of the shaft, the end effector including:
a dilation portion disposed proximate the distal end portion of the shaft,
a transition portion disposed distally on the dilation portion, and
a distal tip portion disposed distally on the transition portion; and
a handle disposed at the proximal end portion of the shaft, the handle including a tip portion steering actuator disposed on the handle;
wherein the tip portion has a tip portion diameter, the dilation portion has a dilation portion diameter, the dilation portion diameter is greater than the tip portion diameter, and the transition portion tapers from the tip portion diameter to the dilation portion diameter between the tip portion and the dilation portion,
the tip portion is movable relative to the dilation portion, and
the tip portion steering actuator enables a user to steer the tip portion by moving the tip portion with the tip portion steering actuator.

2. The dilator of claim 1, further comprising a first tip portion steering line operatively connecting the tip portion and the tip portion steering actuator;
wherein moving the tip portion steering actuator in a first tip portion steering actuator direction causes the tip portion to move in a first tip direction in a tip portion steering plane, and
moving the tip portion steering actuator in the first tip portion steering actuator direction enables a user to steer the tip portion in the first tip direction in the tip portion steering plane.

3. The dilator of claim 2, further comprising a second tip portion steering line operatively connecting the tip portion and the tip portion steering actuator;
wherein moving the tip portion steering actuator in a second tip portion steering actuator direction causes the tip portion to move in a second tip direction in the tip portion steering plane, and
moving the tip portion steering actuator in the second tip portion steering actuator direction enables a user to steer the tip portion in the second tip direction in the tip portion steering plane, the second tip direction being generally opposite the first tip direction in the tip portion steering plane.

4. The dilator of claim 1, wherein the handle further comprises a dilation portion steering actuator disposed on the handle,
the dilation portion is movable relative to the shaft, and
the dilation portion steering actuator enables a user to steer the dilation portion by moving the dilation portion with the dilation portion steering actuator.

5. The dilator of claim 4, further comprising a first dilation portion steering line operatively connecting the dilation portion and the dilation portion steering actuator;
wherein moving the dilation portion steering actuator in a first dilation portion steering actuator direction causes the dilation portion to move in a first dilation direction in a dilation portion steering plane, and
moving the dilation portion steering actuator in the first dilation portion steering actuator direction enables a user to steer the dilation portion in the first dilation direction in the dilation portion steering plane.

6. The dilator of claim 5, further comprising a second dilation portion steering line operatively connecting the dilation portion and the dilation portion steering actuator;
wherein moving the dilation portion steering actuator in a second dilation portion steering actuator direction causes the dilation portion to move in a second dilation direction in the dilation portion steering plane, and
moving the dilation portion steering actuator in the second dilation portion steering actuator direction enables a user to steer the dilation portion in a second dilation direction in the dilation portion steering plane, the second dilation direction being generally opposite the first dilation direction in the dilation portion steering plane.

7. The dilator of claim 1, wherein the tip portion is movable relative to the dilation portion in a tip portion steering plane,
the dilation portion is movable relative to the shaft in a dilation portion steering plane, and
the tip portion steering plane and the dilation portion steering plane are substantially parallel.

8. The dilator of claim 1, wherein the tip portion is movable relative to the dilation portion in a tip portion steering plane,
the dilation portion is movable relative to the shaft in a dilation portion steering plane, and
the dilation portion steering plane is substantially perpendicular to the tip portion steering plane.

9. The dilator of claim 1, further comprising a central lumen extending longitudinally through the handle, the shaft, and the end effector.

10. The dilator of claim 1, wherein the shaft is generally flexible and a stiffness of the shaft varies over a length of the shaft between the proximal end portion and the distal end portion.

11. The dilator of claim 1, further comprising a tip portion energizing element disposed on the tip portion and configured to be operatively connected to a source of electrical energy.

12. The dilator of claim 1, further comprising a dilation portion energizing element disposed on the dilation portion and configured to be operatively connected to a source of electrical energy.

13. The dilator of claim 1, further comprising an expandable element disposed on the dilation portion.

14. The dilator of claim 1, further comprising an expandable element disposed on the tip portion.

15. The dilator of claim 1, further comprising a stent portion removably disposed on the end effector;
wherein the stent portion may be directed to a location by the dilator, and
the dilator may be withdrawn from the stent portion leaving the stent portion in the location.

16. A steerable dilator comprising:
an elongated shaft including a proximal end portion and a distal end portion;
an end effector disposed at the distal end portion of the shaft, the end effector including:
a dilation portion disposed proximate the distal end portion of the shaft, and
a distal tip portion disposed distally on the dilation portion; and
a handle disposed at the proximal end portion of the shaft, the handle including a steering actuator rotatably disposed on the handle;
wherein the tip portion is movable relative to the dilation portion,
the dilation portion is movable relative to the shaft, and
the steering actuator enables a user to steer the tip portion and the dilation portion by moving the tip portion and the dilation portion with the steering actuator.

17. The dilator of claim 16, wherein the steering actuator is generally configured as one or more pivoted supports that allow for the rotation of the steering actuator about a first axis of rotation and a second axis of rotation relative to the handle,
moving the steering actuator in a first actuator direction rotates the steering actuator about the first axis of rotation, and
moving the steering actuator in a second actuator direction rotates the steering actuator about the first axis of rotation, the second actuator direction being generally opposite the first actuator direction.

18. The dilator of claim 17, further comprising:
a first tip portion steering line operatively connecting the tip portion and the steering actuator; and
a second tip portion steering line operatively connecting the tip portion and the steering actuator;
wherein moving the steering actuator in the first actuator direction causes the tip portion to move in a first tip direction in a tip portion steering plane,
moving the steering actuator in the second actuator direction causes the tip portion to move in a second tip direction in the tip portion steering plane,
moving the steering actuator in the first actuator direction enables a user to steer the tip portion in the first tip direction in the tip portion steering plane, and
moving the steering actuator in the second actuator direction enables a user to steer the tip portion in the second tip direction in the tip portion steering plane, the second tip direction being generally opposite the first direction in the tip portion steering plane.

19. The dilator of claim 17, wherein moving the steering actuator in a third actuator direction rotates the steering actuator about the second axis of rotation, and
moving the steering actuator in a fourth actuator direction rotates the steering actuator about the second axis of rotation, the fourth actuator direction being generally opposite the third actuator direction.

20. The dilator of claim 19, further comprising:
a first dilation portion steering line operatively connecting the dilation portion and the steering actuator; and
a second dilation portion steering line operatively connecting the dilation portion and the steering actuator;
wherein moving the steering actuator in the third actuator direction causes the dilation portion to move in a first dilation direction in a dilation portion steering plane,
moving the steering actuator in the fourth actuator direction causes the dilation portion to move in a second dilation direction in the dilation portion steering plane,
moving the steering actuator in the third actuator direction enables a user to steer the dilation portion in the first dilation direction in the dilation portion steering plane, and
moving the steering actuator in the fourth actuator direction enables a user to steer the dilation portion in the second dilation direction in the dilation portion steering plane, the second dilation direction being generally opposite the first dilation direction in the dilation portion steering plane.

21. The dilator of claim 16, wherein the tip portion is movable relative to the dilation portion in a tip portion steering plane,
the dilation portion is movable relative to the shaft in a dilation portion steering plane, and
the tip portion steering plane and the dilation portion steering plane are substantially parallel.

22. The dilator of claim 16, wherein the tip portion is movable relative to the dilation portion in a tip portion steering plane,
the dilation portion is movable relative to the shaft in a dilation portion steering plane, and
the dilation portion steering plane is substantially perpendicular to the tip portion steering plane.

23. The dilator of claim 16, further comprising a central lumen extending longitudinally through the handle, the shaft, and the end effector.

24. The dilator of claim 16, wherein the shaft is generally flexible and a stiffness of the shaft varies over a length of the shaft between the proximal end portion and the distal end portion.

25. The dilator of claim 16, further comprising a tip portion energizing element disposed on the tip portion and configured to be operatively connected to a source of electrical energy.

26. The dilator of claim 16, further comprising a dilation portion energizing element disposed on the dilation portion and configured to be operatively connected to a source of electrical energy.

27. The dilator of claim 16, further comprising an expandable element disposed on the dilation portion.

28. The dilator of claim 16, further comprising an expandable element disposed on the tip portion.

29. The dilator of claim 16, further comprising a stent portion removably disposed on the end effector;
wherein the stent portion may be directed to a location by the dilator, and
the dilator may be withdrawn from the stent portion leaving the stent portion in the location.

30. The dilator of claim 16, further comprising a light element disposed on the tip portion and configured to be operatively connected to a source of light.

31. A steerable catheter comprising:
an elongated shaft including a proximal end portion and a distal end portion;
an end effector disposed at the distal end portion of the shaft, the end effector including:
a proximal end portion disposed proximate the distal end portion of the shaft, and
a distal tip portion disposed distally on the end effector; and
a handle disposed at the proximal end portion of the shaft, the handle including a steering actuator rotatably disposed on the handle;
wherein the tip portion is movable relative to the shaft;
the proximal end portion of the end effector is movable relative to the shaft; and
the steering actuator enables a user to steer the tip portion and the proximal end portion of the end effector by moving the tip portion and the proximal end portion of the end effector with the steering actuator.

32. The catheter of claim 31, wherein the steering actuator is generally configured as one or more pivoted supports that allow for the rotation of the steering actuator about a first axis of rotation and a second axis of rotation relative to the handle,
moving the steering actuator in a first actuator direction rotates the steering actuator about the first axis of rotation, and
moving the steering actuator in a second actuator direction rotates the steering actuator about the first axis of rotation, the second actuator direction being generally opposite the first actuator direction.

33. The catheter of claim 32, further comprising:
a first tip portion steering line operatively connecting the tip portion and the steering actuator; and
a second tip portion steering line operatively connecting the tip portion and the steering actuator;
wherein moving the steering actuator in the first actuator direction causes the tip portion to move in a first tip direction in a tip portion steering plane,
moving the steering actuator in the second actuator direction causes the tip portion to move in a second tip direction in the tip portion steering plane,
moving the steering actuator in the first actuator direction enables a user to steer the tip portion in the first tip direction in the tip portion steering plane, and
moving the steering actuator in the second actuator direction enables a user to steer the tip portion in the second tip direction in the tip portion steering plane, the second tip direction being generally opposite the first direction in the tip portion steering plane.

34. The catheter of claim 32, wherein moving the steering actuator in a third actuator direction rotates the steering actuator about the second axis of rotation, and
moving the steering actuator in a fourth actuator direction rotates the steering actuator about the second axis of rotation, the fourth actuator direction being generally opposite the third actuator direction.

35. The catheter of claim 34, further comprising:
a first end effector steering line operatively connecting the end effector and the steering actuator; and
a second end effector steering line operatively connecting the end effector and the steering actuator;
wherein moving the steering actuator in the third actuator direction causes the end effector to move in a first end effector direction in an end effector steering plane,
moving the steering actuator in the fourth actuator direction causes the end effector to move in a second end effector direction in the end effector steering plane,
moving the steering actuator in the third actuator direction enables a user to steer the end effector in the first end effector direction in the end effector steering plane, and
moving the steering actuator in the fourth actuator direction enables a user to steer the end effector in the second end effector direction in the end effector steering plane, the second end effector direction being generally opposite the first end effector direction in the end effector steering plane.

36. The catheter of claim 31, wherein the tip portion is movable in a tip portion steering plane,
the proximal end portion of the end effector is movable in an end effector steering plane, and
the tip portion steering plane is substantially parallel to the end effector steering plane.

37. The catheter of claim 31, wherein the tip portion is movable in a tip portion steering plane,
the proximal end portion of the end effector is movable in an end effector steering plane, and
the tip portion steering plane is substantially perpendicular to the end effector steering plane.

38. The catheter of claim 31, further comprising a central lumen extending longitudinally through the handle, the shaft, and the end effector.

39. The catheter of claim 31, wherein the shaft is generally flexible and a stiffness of the shaft varies over a length of the shaft between the proximal end portion and the distal end portion.

40. The catheter of claim 31, further comprising a tip portion energizing element disposed on the tip portion and configured to be operatively connected to a source of electrical energy.

41. The catheter of claim 31, further comprising a light element disposed on the tip portion and configured to be operatively connected to a source of light.

42. A method of creating an opening in a biological tissue membrane, the method comprising:
advancing an end effector of a dilator to proximate a biological tissue membrane by steering a tip portion in a tip portion steering plane;
creating an initial opening through the membrane by advancing the tip portion of the end effector through the membrane;
expanding the initial opening to an expanded opening by advancing at least a portion of a dilation portion of the end effector through the membrane; and
withdrawing the end effector from the expanded opening.

43. A method of applying light to brain and/or spinal cord tissue, the method comprising:
advancing an end effector of a dilator to proximate the brain and/or spinal cord tissue; and
applying light to the brain and/or spinal cord tissue.
